(19) United States
(12) Patent Application Publication
(10) Pub. No.: US 2005/0143793 A1
(43) Pub. Date: Jun. 30, 2005

(54) Phototherapy for psoriasis and other skin disordes

(76) Inventors: Avner Korman, Herzelia (IL); Yoram Harth, Haifa (IL); Joshua Degani, Jerusalem (IL)

Correspondence Address: WELSH & KATZ, LTD, 120 S RIVERSIDE PLAZA, 22ND FLOOR, CHICAGO, IL, 60606, US

(21) Appl. No.: 10/497,483

(22) PCT Filed: Dec. 5, 2002
(86) PCT No.: PCT/IL02/00980

(30) Foreign Application Priority Data
Dec. 6, 2001 (IL) 14664
Feb. 19, 2002 (IL) 148257

(51) Int. Cl. A61N 1/00

(52) U.S. Cl. 607/94

(57) ABSTRACT

Single-core and multi-core microcapsules are provided, having multiple shells, at least one of which is formed of a complex a coacervate of two components of shell materials. The complex coacervate may be the same or different for each shell. Also provided are methods for making the microcapsules.

### FIELD OF THE INVENTION

The present invention relates to an apparatus, a system and a method for the phototherapy of psoriasis and other skin disorders.

### BACKGROUND OF THE INVENTION

Psoriasis is a non-contagious skin disorder that most commonly appears as inflamed swollen skin lesions covered with silvery white scale. This most common type of psoriasis is called “plaque psoriasis.” Psoriasis comes in many different variations and degrees of severity. Different types of psoriasis display characteristics such as pus-like blisters (pustular psoriasis), severe sloughing of the skin (erythrodermic psoriasis), drop-like dots (guttate psoriasis) and smooth inflamed lesions in the flexural areas (inverse psoriasis). The degrees of severity of psoriasis ate divided into three important categories: mild, moderate and severe.

Skin cells are programmed to follow two possible programs: normal growth or wound healing. In a normal growth pattern, skin cells are created in the basal cell layer, and then move up through the epidermis to the stratum corneum, the outermost layer of the skin. Dead cells are shed from the skin at about the same rate as new cells are produced, maintaining a balance. This normal process takes about 28 days from cell birth to death. When skin is wounded, a wound healing program, also known as regenerative maturation, is triggered. Cells are produced at a much faster rate, theoretically to replace and repair the wound. There is also an increased blood supply and localized inflammation. In many ways, psoriatic skin is similar to exaggerated skin healing from a wound or reacting to a stimulus such as infection.

Lesional psoriasis is characterized by cell growth in the alternate growth program. Although there is no wound at a psoriatic lesion, skin cells, also referred to as keratinocytes, behave as if there is. These keratinocytes switch from the normal growth program to regenerative maturation. Cells are created and pushed to the surface in as little as 2-4 days, and the skin cannot shed the cells fast enough. The excessive skin cells build up and form elevated, scaly lesions. The white scale (called “plaque”) that usually covers the lesion is composed of dead skin cells, and the redness of the lesion is caused by increased blood supply to the area of rapidly dividing skin cells.

It is known that ultraviolet (UV) light can suppress the inflammation in psoriatic plaques and decrease the abnormal proliferation of epidermal keratinocytes by affecting the DNA molecules of the radiated cells.

Phototherapy using ultraviolet light in general and specifically UVB light, is a well-known and accepted treatment for widespread psoriasis. Typically, the whole or a large part of the body of the psoriatic patient is irradiated by UV light (in most cases UVB—250-340 nm). The light is commonly generated by apparatus based on an array/matrix of UV emitting fluorescent light bulbs at relatively low UVB energy flux. Usually 30-40 such treatments are needed for the clearing of skin lesions. These current methods of psoriasis therapy have the disadvantage that that the entire skin, including “healthy” skin parts, is exposed to harmful UV rays, and a long session of treatment is required to get only partial relief of symptoms.

Basic science and clinical research have shown that light with a narrow band in the wavelength range of 300-313 nm is most effective for psoriasis therapy. The rationale for this UVB source stems from elucidation of the UV action spectrum for clearing of psoriasis. In two studies, it was determined that UV wavelengths (“mercury lines”) longer than 313 nm were not effective in resolving psoriasis, whereas wavelengths shorter than 300 nm produced only UV burns (erytherna) without clearing psoriasis. Further dissection of wavelengths from 300 to 313 nm established an “optimal” therapeutic index at 313 nm (defined as the lowest fraction of a Minimal Erythema Dose [MED] required to remit psoriatic skin lesions). Subsequently, a 311-nm UVB fluorescent bulb-based source was developed for treatment of psoriasis. Several European studies have established the effectiveness of this treatment when used 3 to 5 times a week at erythemogenic doses. In fact, narrow band UVB (NB-UVB) has proved to be even more effective than the modified Goeckerman regimen when used on a daily basis in patients with severe recalcitrant psoriasis (even in patients with highly pigmented skin). However, full-body aggressive administration of NB-UVB at erythemogenic or near-erythemogenic levels proved to be difficult because of somewhat unusual burning responses to this light source. Typical clearing regimen treatment with full-body narrow band fluorescent bulbs (TL-01) employs a cumulative dose of 13 J/cm² (range 4 J/cm²-24 J/cm²). Clearing is achieved in 80% of treated patients in 4 weeks, 3 treatments/week, with 50% of patients remaining clear after 6 months.

Hartman describes apparatus for targeted UV phototherapy of skin disorders, based on a UV arc lamp, in U.S. Pat. No. 6,413,268. The arc lamp is housed in a base unit, which has one or more output ports to which a flexible optical guide may be connected. The light from the arc lamp is directed into the optical guide, which conveys the light to a handpiece at its opposite end. The handpiece is used to deliver a shaped beam of radiation to a target area of a patient. In one embodiment, the base unit has two output ports, one for UVB radiation, and the other for UVA radiation. Optical guides may be connected to one or both of the ports for delivering the desired type of radiation to the skin.

It is known that normal skin can be exposed to up to 3 MEDs without blistering while psoriatic skin may be exposed to up 3 times that dose without blistering. A therapy targeted only on psoriasis plaques, sparing the “healthy” skin, may thus employ higher fluences and may shorten time to clearance. Another advantage of targeted phototherapy may be the sparing of non-psoriatic skin from harmful UV effects. Recently Asawanonda et al. treated psoriasis with an excimer laser emitting 308 nm. The MED in their patients was 203 mJ/cm². They report that medium fluences at 2-6 MED (400-1200 mJ/cm²) may result in clearing after 2 weeks at 2 sessions a week. Fluences of up to 6 MEDs on psoriasis plaques were well tolerated by all patients. Most patients remained cleared after 2 months of follow-up.

Lasers are used by dermatologists for a variety of procedures. Increasingly, treating psoriasis with some types of lasers can be an option for both physicians and patients to consider, but has its limitations and operational constraints, as well as high purchase costs.

A device called X-trac™ is an excimer laser-based system, manufactured by Photomedex of Radnor, Pa. It was approved by the U.S. Food and Drug Administration (FDA) for psoriasis treatment, in early 2000. Other types of excimer laser-based systems have also received FDA approval. The laser emits a high-intensity beam of UV light at a wavelength of 308 nm, close to the light delivered by conventional narrow-band UVB units. The main disadvantages of this system are its narrow, high peak power pulses, high purchasing and operational costs, complicated and expensive maintenance and low reliability. Also, the system's large physical size is a limit in the small dermatologist's typical treatment room.

The largest study of X-trac enrolled 124 patients, and 80 patients completed the study, meaning the treatment cleared their psoriasis or they had 10 treatments. Of those, 72 percent achieved at least 75 percent improvement in their psoriasis, after an average of 6.2 treatments. At least 90 percent improvement was seen in 35 percent of the patients, after an average of 7.5 treatments. How well an individual will respond to the treatment varies. Photomedex reports that it takes an average of 4 to 10 sessions to see results, depending on the particular case of psoriasis. Skin with psoriasis can handle much more UT light than unaffected skin. Therefore, higher doses can be used with the laser compared to traditional UVB units. For those who respond, this should mean quicker results: 10 or fewer laser sessions vs. 30 to 40 treatments for regular UVB.

Selective photothermolysis is a method, described by Anderson and Parrish in 1983 (“Selective Photothermolysis: Precise Microsurgery by Selective Absorption of Pulsed Radiation,” Science, Vol. 220, pp. 524-527), for destroying certain diseased or unsightly tissue, on or near the skin, with minimal damage to the surrounding healthy tissue. The tissue to be destroyed is generally characterized by significantly greater optical absorption at some wavelength of electromagnetic radiation than the surrounding tissue. The prior art methods include irradiating the target and the surrounding tissue with pulsed light radiation, usually visible radiation that is preferentially absorbed by the target. The energy and duration of the pulses is such that the target is heated to between about 70 C and about 80 C, at which temperature the proteins of the target coagulate. Because the target absorbs the incident radiation much more strongly than the surrounding tissue, the surrounding tissue absorbs much less heat and, for at least short periods of exposure, does not reach a temperature to cause damage. However, the surrounding healthy tissue must be prevented from heating up over an extended heating period.

Usually, the radiation source used in photothermolysis is a laser, for example a flash lamp-pulsed dye laser. A laser source has the advantage of being inherently monochromatic. Other sources include broadband sources used in conjunction with narrow band filters, as described, for example, by Gustaffson in PCT patent publication WO 91/15264. A similar device, called the “Photoderm-VL,” is manufactured by Lumenis Ltd. of California. Suitable targets for selective photothermolysis include general sub-surface veins, as well as birthmarks, port-wine stains, spider veins, and varicose veins, all of which tend to be much redder than the surrounding tissue because of their higher concentration of oxyhemoglobin-containing red blood cells.

Anderson and Parrish used light of a wavelength of 577 nm, corresponding to the 577 nm oxyhemoglobin absorption band. It was subsequently determined (Tian, Morrison, and Kurban, “585 nm for the Treatment of Port-Wine Stains,” Plastic and Reconstructive Surgery, vol. 86 no. 6 pp. 1112-1117) that 585 nanometers is a more effective wavelength to use.

One constraint on the pulse duration used in photothermolysis is that the surrounding tissue must not be heated to the point that it, too, begins to coagulate. As the disorder (hereinafter sometimes referred to as the “target”) is heated, heat is conveyed by convection and conduction from the target to the cooler surrounding tissue. To keep the surrounding tissue from being heated to the point of damage, the pulse length in the prior art is kept on the order of the target's thermal relaxation time. For relatively small targets, such as birthmarks, port-wine stains, and spider veins, typical pulse lengths are on the order of hundreds of microseconds. For varicose veins, pulse lengths on the order of milliseconds should be used.

Selective photothermolysis also has been used to treat psoriatic skin tissue. Flash-lamp-pumped pulsed dye laser beams have been used to selectively destroy cutaneous blood vessels. Light passing through the epidermis is preferentially absorbed by hemoglobin, the major chromophore in the blood of the ectatic capillaries of the upper dermis. The radiant energy is converted to heat causing thermal damage and necrosis in the target. Flash-lamp-pumped pulsed dye laser radiation in general destroys the targeted dermal disorder. The problem is the prevention of damage to the surrounding healthy tissue. For example, port wine stains are known to be characterized by normal epidermis overlying an abnormal plexus of dilated blood vessels located on a layer in the upper dermis.

The predominant endogenous and/or cutaneous chromophores that absorb light at the 585 nm wavelength produced by flash-lamp-pumped pulsed dye laser are melanin and hemoglobin. Accordingly, the overlying epidermal pigment layer acts as an optical shield through which the light must pass to reach the underlying lesion such as those caused by port wine stain blood vessels. The absorption of laser energy by the melanin causes localized heating in the epidermis and reduces the light dosage reaching the target thereby decreasing the quantity of heat in the targeted area, leading to sub-optimal blanching of the tissue disorder or necessitating increased time periods of treatment with consequent increased risk of healthy tissue damage, unless steps are taken to protect the healthy tissue.

Prior art cooling methods used to prevent damage to healthy tissue include the use of lens-like contact devices having high thermal conductivity and having a refractive index that enables the optical radiation to be coupled to the epidermis, i.e., a refractive index of approximately 1.55. Thus, the contact device is preferably formed of a high-density material such as sapphire or other similar optically transparent glass or plastic. See, for example, U.S. Pat. No. 5,595,568, the disclosure of which is incorporated herein by reference.

### SUMMARY OF THE INVENTION

There is thus a widely-recognized need for an effective way to treat areas affected by skin disorders, such as psoriatic plaques, vitiligo, and atopic dermatitis, and other localized skin disorders such as keloids and stretch marks scars with a well-concentrated UV light source. Such a UV source should overcome the limitations of both low energy-flux fluorescent-based devices and of the integrated energy of a train of short pulses generated by high peak-power UVB laser-based devices. The source should provide a homogenous, comparatively small exposure area, well-defined beam profile with output energy within a prescribed narrow spectral range.

Some embodiments of the present invention offer solutions to this need based on simple and reliable types of UVB-emitting sources, having a high energy concentration and operating in CW mode or with long-pulse duration. These solutions provide the user with a constant UVB power level during the entire exposure time. The treatment methods and related systems, in accordance with these embodiments of the present invention, are simple and economical, enabling easy operation and minimal maintenance procedures in the clinic, using low-cost yet reliable apparatus.

In other embodiments of the present invention, a system for treating multiple skin disorder indications generates output energy in one or several narrow spectral bands, selectable from UVB, UVA and violet bands. The energy may be generated by a single light source or multiple light sources, having a high energy concentration and operating in CW mode or with long-pulse duration. These embodiments allow the user to electrically select a narrow UVB, UVA or violet treatment band, or a combination of different bands, with constant power level during the entire exposure time. The selected band outputs are typically conveyed through a flexible light guide to the patient's skin. The system is preferably configured so that a single light guide can be used to convey the radiation in any or all of the bands, regardless of which bands are selected. This system thus provides a simple, economical, electrically or electromechanically selectable set of dose and spectral energy exposure treatments, using a single treatment unit, which is easy to operate and requires minimal maintenance.

In another embodiment, the UVB radiation-based treatment is combined with a photothermolysis treatment effect induced by a short-duration pulsed secondary light source.

In yet another embodiment, the output energy pattern of the apparatus and associated marking means provide the operator with the ability to cover homogeneously, in a matrix registration mode, the entire affected skin area, while not affecting or damaging the non-lesional “healthy skin” around the psoriatic skin area.

The present invention thus provides an effective phototherapy method that is simple and economical to operate and is expected to effectively clear psoriasis, vitiligo, atopic dermatitis and other skin disorders in a small number of treatment sessions, with minimal after-effects. The required solution replaces commonly-used fluorescent-based treatment systems, which typically require 30-40 treatments, and laser systems, which are costly and difficult to maintain. In addition, the present invention may be used to treat conditions such as localized scleroderma, localized cutaneous T-cell lymphoma and hypopigmented scars.

There is therefore provided, in accordance with an embodiment of the present invention, apparatus for treatment of psoriasis and other skin disorders, including:
a radiation source, adapted to generate ultraviolet B (UVB) radiation suitable for treatment of a psoriatic plaque; and
radiation delivery optics, coupled to the radiation source so as to concentrate and deliver the generated UVB radiation to the plaque with intensity of at least 75 mJ/cm² delivered to the plaque over a period of less than 10 sec, and with a spectral width of at least 30 nm, so as to engender clearing of the plaque.

In an embodiment of the invention, the radiation source further includes a source of visible radiation suitable to photothermolyze blood vessels in a vicinity of the plaque.

Typically, the radiation source includes a non-coherent UV source, which is adapted to generate the UVB radiation continuously, wherein the non-coherent UV source includes at least one of a group of sources consisting of a metal halide gas discharge lamp and an excimer lamp. Additionally or alternatively, the radiation source includes a pulsed source emitting at least one of visible radiation and near infrared (IR) radiation.

There is also provided, in accordance with an embodiment of the present invention, apparatus for treatment of a skin condition, including:
a radiation source, adapted to irradiate an area of the skin with radiation in at least one of an ultraviolet, visible and infrared spectral range; and
marking means, adapted to delineate the area on the skin responsive to irradiation of the area by the radiation source.

Typically, the marking means includes one or more markers, adapted to print a plurality of marks on the skin delineating the irradiated area, and the apparatus includes an arm supporting the radiation source, and an imaging device coupled to capture an image of the skin, and a processor adapted to analyze the marks on the skin appearing in the image so as to guide the radiation source by controlling movement of the arm, responsive to the marks in the image.

Alternatively, the radiation source includes a radiation guide, which is brought into proximity with the area of the skin so as to deliver the radiation thereto, and the marking means is adapted to mark a periphery of the radiation guide.

The marking means may include a photosensitive substance, which is applied to the skin prior to irradiating the area of the skin, wherein the radiation causes a visible change in the photosensitive substance, thereby delineating the area.

There is additionally provided, in accordance with an embodiment of the present invention, apparatus for treatment of psoriasis, including:
a radiation source, adapted to irradiate a psoriatic area of skin with ultraviolet (UV) radiation, so as to treat the psoriasis;
an optical sensor, adapted to detect an optical quality of the irradiated area; and
a dosage controller, coupled to receive an indication of the optical quality from the optical sensor and to control the radiation source responsive to the indication.

Typically, the optical sensor is adapted to sense a change in at least one of a color and a texture of the irradiated area indicative of erytherna resulting from the radiation, and the dosage controller is adapted to control a flux of the radiation applied to the skin by the radiation source so as to engender a desired level of the erythema.

There is further provided, in accordance with an embodiment of the present invention, a method for treatment of psoriasis, including:
applying ultraviolet B (UVB) radiation to a psoriatic plaque; and
applying visible radiation suitable to photothermolyze blood vessels in a vicinity of the plaque, substantially simultaneously with applying the UVB radiation.

Typically, the blood vessels photothermolyzed by the radiation in the vicinity of the plaque include blood vessels under the plaque.

There is moreover provided, in accordance with an embodiment of the present invention, a method for treating a skin condition, including:
irradiating an area of the skin with radiation in at least one of an ultraviolet, visible and infrared spectral range; and
marking the area on the skin responsive to irradiation of the area by the radiation source, so as to provide an indication of the area of the skin that was treated.

There is furthermore provided, in accordance with an embodiment of the present invention, a method for treatment of psoriasis, including:
irradiating a psoriatic area of skin with ultraviolet (UV) radiation, so as to treat the psoriasis;
detecting an optical quality of the irradiated area; and
controlling a level of the radiation responsive to the indication.

The method may include, prior to irradiating the psoriatic area of the skin, applying a quickly-solidifying, self-peeling cream or gel material, having UV energy protection capability including at least one of absorption and reflection properties, to a healthy skin area on a periphery of the psoriatic area.

There is also provided, in accordance with an embodiment of the present invention, apparatus for treatment of skin disorders, including:
a radiation source, adapted to generate ultraviolet (UV) radiation suitable for treatment of skin affected by one or more of the skin disorders; and
radiation delivery optics, coupled to the radiation source so as to concentrate and deliver the generated UV radiation directly to the affected skin with an energy flux level per single treatment and specific affected skin area selected from an energy radiation flux group consisting of at least a radiation flux equal or higher than 50 mJ/cm² in less than 30 sec per treatment and a radiation flux higher than 1.5 MED (Minimum Erythema Dose),
wherein the UV radiation is in a spectral range of 296-390 nm and includes at least one spectral line of substantial intensity having a bandwidth of at least 1 nm, wherein the spectral line is selected so as to engender clearing of the affected skin.

Typically, the radiation source includes a non-coherent UV source, which is adapted to generate the UVB radiation continuously during a predefined exposure time. Preferably, the UV radiation includes a radiation band in the spectral range of 296-313 nm, and the radiation band includes multiple spectral lines, which are chosen so as to enhance an efficacy of the treatment. More preferably, the spectral line is within the spectral range of 296-305 nm, and the spectral line is chosen so as to facilitate the efficacy of the treatment by at least one of maximizing treatment effect and minimizing treatment time of the affected skin area. Most preferably, the spectral line emission is within the spectral range of 300-304 nm.

In embodiments of the present invention, the non-coherent radiation source includes at least one of a group of sources consisting of a metal halide gas discharge lamp, a laser diode matrix, a light emitting diode (LED) matrix, and an excimer lamp.

Typically, the radiation delivery optics are adapted to limit an exposure area of the radiation to the affected skin, wherein the radiation delivery optics include one of a selection of interchangeable light guides, which are adapted to direct the radiation toward the affected skin during the treatment, and wherein the light guides are selectable so as to match an output aperture of the selected light guide to a size and shape of the affected skin. In an embodiment of the present invention, the output apertures of the interchangeable light guides are selected from a group including aperture areas of at least 200 mm², 400 mm² and 2500 mm². Additionally or alternatively, the radiation delivery optics further include aperture shaping optics at the output aperture so as to provide an aperture shape that is adapted for optimal plaque area energy coverage.

In an embodiment of the invention, a module containing the radiation source and the radiation delivery optics, and an articulated arm, which is coupled to suspend and position the module relative to the affected skin.

Typically, the radiation delivery optics include an optical filter, which is adapted to limit the spectral range of energy emitted by the light source is limited to the spectral range above 296 nm. Additionally or alternatively, the radiation delivery optics include an optical bandpass filter, which is adapted to limit the spectral range of energy emitted by the light source to the spectral range above 296 nm and under 390 nm.

In embodiments of the invention, the radiation source is adapted to provide a continuous energy output during treatment of the affected skin, with a momentary output peak power smaller than 10 kW/cm². The radiation source may includes a metal halide lamp, whose output spectrum includes spectral lines at 303, 306, 308, 309, and 312 nm.

In an embodiment of the invention, the apparatus is adapted to determine the radiation flux corresponding to the MED by determining a minimum energy dosage creating erytherna on normal skin in a vicinity of the affected skin area, whereby operating parameters of the apparatus are set to the radiation flux thus determined.

There is additionally provided, in accordance with an embodiment of the present invention, a lamp for treatment of skin disorders, the lamp including:
a transparent envelope;
a mixture of species contained within the envelope, the species including a halogen and a plurality of metals selected from a group of metals consisting of mercury, bismuth, aluminum, cesium, iron and gallium; and
discharge electrodes disposed within the envelope, so as to generate an arc within the mixture of species, thereby causing emission of ultraviolet radiation.

Typically, the species are pressurized within the envelope, so that while the discharge electrodes are generating the arc, a gas pressure within the envelope is greater than 5 atm.

There is further provided, in accordance with an embodiment of the present invention, apparatus for treatment of skin disorders, including:
a radiation source, which is adapted to generate radiation in multiple spectral bands;
a radiation guide, which is optically coupled to receive the radiation in all of the multiple spectral bands, and to convey the received radiation to an area of skin affected by one of the disorders, so as to treat the affected area; and
a band selector, which is adapted to select one or more of the multiple spectral bands to be conveyed by the radiation guide, in response to a therapeutic indication.

Typically, the radiation source includes a metal halide arc lamp, wherein the metal halide arc lamp is a high-pressure lamp, containing at least one of bismuth, cesium, iron and gallium.

The multiple spectral bands may include at least one band in each of an ultraviolet A (UVA) range, an ultraviolet B (UVB) range, and a visible range. In embodiments of the invention, the visible range includes a violet light range and/or a red light range.

In an embodiment of the invention, the band selector is adapted to select at least two of the spectral bands to be conveyed by the radiation guide simultaneously, wherein the at least two of the bands include the at least one band in one of the UVA and UVB ranges and the at least one band in the visible range. For example, the at least one band in the one of the UVA and UVB ranges may include one or more wavelengths in the UVB range that provide effective treatment of a psoriatic plaque, while the at least one band in the visible range includes violet light suitable for treating inflammation associated with the plaque.

In another embodiment, the radiation source includes a plurality of lamps, each operating in one or more of the spectral bands, and a beam combiner for combining the radiation from the plurality of the lamps to be received by the radiation guide. The plurality of lamps may include electrical discharge lamps and/or solid-state light sources. Typically, the beam combiner includes one or more dichroic mirrors, which are adapted to selectively reflect the radiation emitted by the lamps. Additionally or alternatively, the band selector is arranged to actuate one or more of the lamps to operate so as to provide the selected one or more of the spectral bands.

Further additionally or alternatively, the band selector includes one or more optical filters, having a selectable spectral passband.

In embodiments of the invention, the radiation guide includes at least one of a fiberoptic light guide and a liquid-filled light guide.

The radiation guide includes a proximal end, which is coupled to receive the radiation, and a distal end, which is adapted to deliver the radiation to the area of the skin, and the apparatus may further include:
a receptacle, for receiving the distal end of the radiation guide;
a detector, which is coupled to receive the radiation emitted from the distal end of the radiation guide when the radiation guide is inserted in the receptacle, and to generate a signal in response to an intensity of the radiation; and
a controller, which is coupled to receive the signal from the detector and to determine, based on the signal, an output level of the apparatus.

Typically, the controller is adapted to adjust an operating level of the radiation source in response to the signal, so as to adjust the output level to a predetermined value.

In a further embodiment, the apparatus includes a controller having a user interface, which is operable by an operator of the apparatus to input the therapeutic indication to the band selector. The user interface may be further operable by the operator to initiate a procedure, using the apparatus, for determining a Minimal Erythema Dose (MED) of a patient in treatment, and the controller is adapted to set a level of the radiation to be applied to the affected area of the skin of the patient based on the determined MED. Typically, the controller includes a memory, and the user interface is further operable by the operator to record and recall a treatment history of a patient in treatment using the apparatus.

There is moreover provided, in accordance with an embodiment of the present invention, a method for treatment of skin disorders, including:
directing radiation in multiple spectral bands toward an input end of a radiation guide;
selecting one or more of the multiple spectral bands to be input to the radiation guide through the input end, in response to a therapeutic indication with respect to an area of skin affected by one of the disorders; and
applying an output end of the radiation guide to the affected area of the skin as to treat the affected area.

The present invention will be more fully understood from the following detailed description of the embodiments thereof taken together with the drawings in which:

### BRIEF DESCRIPTION OF THE DRAWINGS

FIG. 1 is a schematic, pictorial illustration of a system for phototherapy, in accordance with one embodiment of the present invention;

FIG. 2 is a schematic, internal view of an optical head used in phototherapeutic irradiation of the skin, in accordance with one embodiment of the present invention;

FIG. 3 is a block diagram that schematically illustrates circuitry used in controlling phototherapeutic energy dosage, in accordance with another embodiment of the present invention;

FIG. 4A is a schematic side view of phototherapeutic apparatus with automated registration of the treated area, in accordance with a one embodiment of the present invention;

FIG. 4B is a schematic top view of skin of a patient that has been treated using the apparatus of FIG. 4A;

FIG. 5A is a schematic cross-sectional side view of optics, including an elliptical reflector and light guide, used to concentrate phototherapeutic radiation on the skin of a patient, in accordance with another embodiment of the present invention;

FIG. 5B is a schematic optical diagram showing details of the elliptical reflector of FIG. 5A;

FIG. 5C is a schematic, pictorial illustration of the light guide of FIG. 5A;

FIG. 6A is a schematic, pictorial illustration of an optical head used in dual-band phototherapy, in accordance with one embodiment of the present invention;

FIG. 6B is a schematic side view of an optical head used in dual-band phototherapy, in accordance with another embodiment of the present invention;

FIG. 6C is a schematic side view of an optical head used in dual-band phototherapy, in accordance with yet another embodiment of the present invention;

FIG. 7 is a plot that schematically illustrates spectral lines and bands used in dual-band phototherapy, in accordance with another embodiment of the present invention;

FIG. 8 is a plot that schematically illustrates a spectral line and associated spectral band used in UVB phototherapy, in accordance with one embodiment of the present invention;

FIG. 9A is a schematic cross-sectional side view of an excimer lamp illumination source that is integrated within an optical head used in UVB phototherapy, wherein the output energy is directed perpendicular to the mechanical axis of the lamp, in accordance with another embodiment of the present invention;

FIG. 9B is a schematic cross-sectional side view of an excimer lamp illumination source that is integrated within the optical head used in UVB phototherapy, wherein the output energy is directed alongside the mechanical axis of the lamp, in accordance with another embodiment of the present invention;

FIG. 10 is a schematic cross-sectional side view of an optical head used in UVB phototherapy based on an excimer lamp, in accordance with yet another embodiment of the present invention.

FIGS. 11A and 11B are schematic, pictorial illustrations of systems for phototherapy, in accordance with further embodiments of the present invention;

FIG. 11A is a schematic, pictorial illustration of a system for phototherapy in a use mode, in accordance with another embodiment of the present invention;

FIG. 11B is a schematic, pictorial illustration of a system for phototherapy in a standby mode, in accordance with another embodiment of the present invention;

FIG. 12A is a schematic cross-sectional side view of optics used in a system for phototherapy, in accordance with an embodiment of the present invention;

FIG. 12B is a schematic, pictorial illustration showing further details of the optics of FIG. 12A;

FIG. 12C is a schematic optical diagram showing optics used in a system for phototherapy, in accordance with another embodiment of the present invention;

FIGS. 12D and 12E are schematic optical diagram showing optics used in systems for multi-band phototherapy, in accordance with other embodiments of the present invention;

FIG. 13 is a schematic, pictorial, partly cutaway illustration showing details of an optical treatment head and receptacle used in a system for phototherapy, in accordance with an embodiment of the present invention;

FIGS. 14 and 15 are plots that schematically illustrate output spectral bands of a system for phototherapy, in accordance with embodiments of the present invention; and

FIGS. 16A-16E are schematic representations of computer screens used in a graphical user interface of a system for phototherapy, in accordance with an embodiment of the present invention.

### DETAILED DESCRIPTION OF PREFERRED EMBODIMENTS

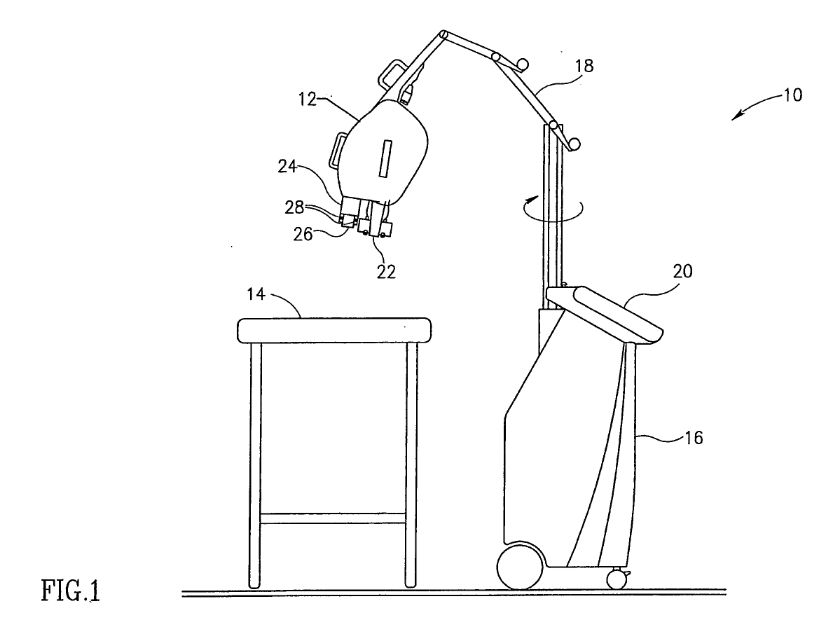

FIG. 1 is a schematic, pictorial illustration of a system 10 for phototherapeutic treatment of psoriasis and other skin disorders, in accordance with one embodiment of the present invention. System 10 comprises an optical head 12, which is used to irradiate the skin of a patient (not shown), who typically lies on a treatment table 14. Details of optical head 12, in a number of different, alternative embodiments, are shown in the figures that follow. Optical head 12 is connected to a power supply and control console 16 by an adjustable suspension arm 18, which allows an operator of system 10 to adjust the vertical, horizontal and radial placement of the optical head relative to the patient's body.

Adjustable suspension arm 18 can be made for pure mechanical movement, to be manipulated by an operator of head 12, the arm having integrated balancing weights on each arm section. Alternatively, a balancing counter-tension spring is integrated into at least one of the sections of arm 18. In another embodiment an electromechanical movement control unit, such as a motor, is integrated into the movement axis of each of the sections of arm 12, so as to enable electrical control of the head position in space. Thus, the vertical, horizontal and radial placement of the optical head are adjusted relative to the patient's body. In one embodiment of system 10, the movement and positioning control of unit 12 is done by an embedded computer in control console 16, wherein the positioning of head 12 is defined by the computerized analysis of the image of the treated affected area, as visualized by a camera unit 24. Console 16 includes a control panel 20, enabling the operator to adjust the position of head 12, as well as to control treatment parameters; to operate, generate and digitally store images using camera unit 24; and to receive feedback from system 10.

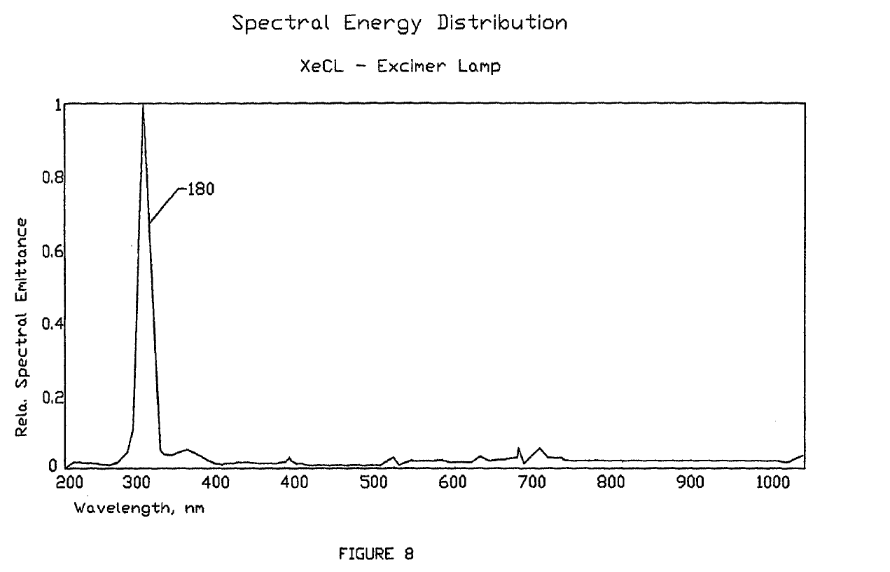

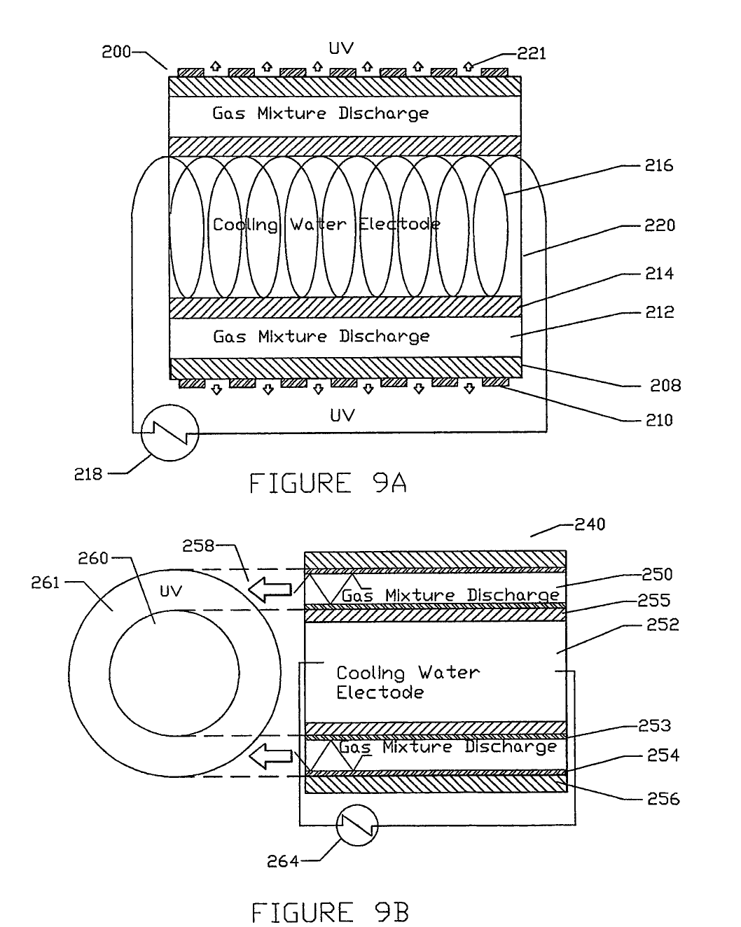

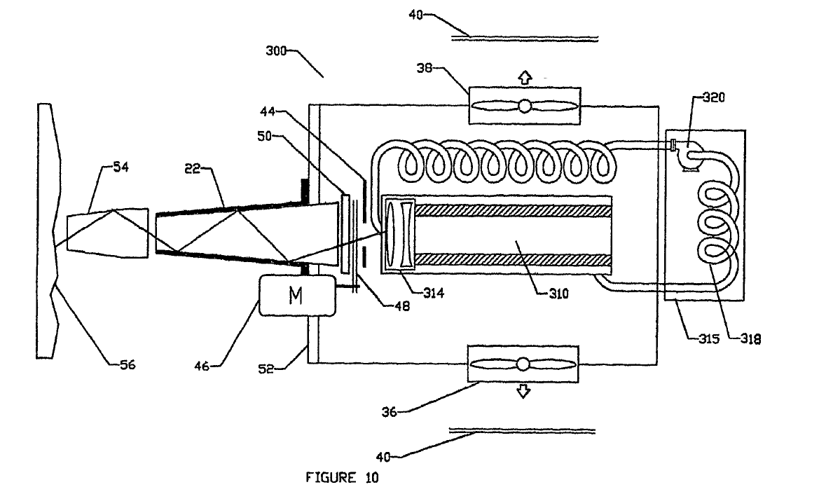

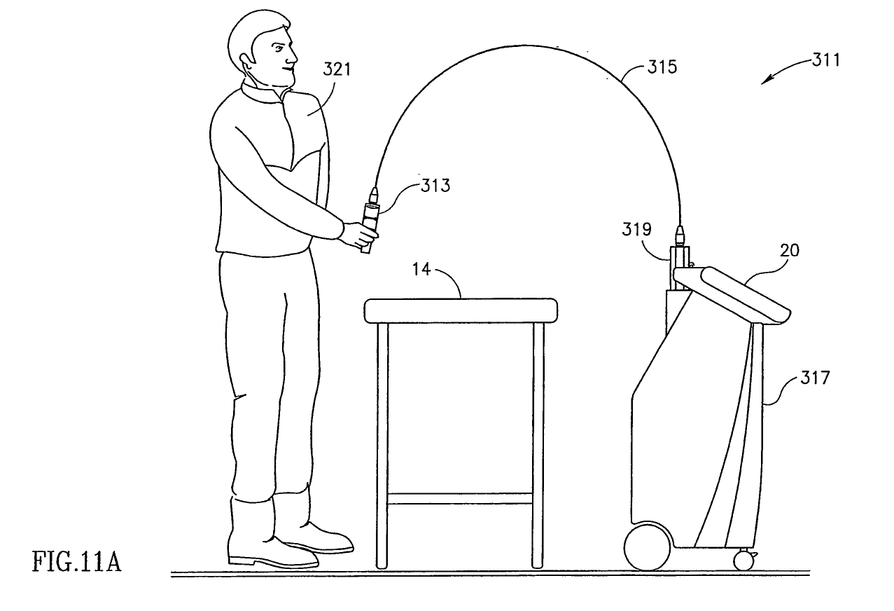

Optical head 12 preferably generates intense, ultraviolet (UV) radiation, which is conveyed to a treatment area on the patient's skin by a light guide 22. Preferably, the radiation comprises broadband, continuous-wave (CW), non-coherent UVB radiation, which is applied at dosages above the MED threshold, sufficient to clear psoriatic plaques. (Here “broadband” refers to radiation bandwidth greater than about 30 nm.) Alternatively, system 10 and optical head 12 may be used to apply narrowband and/or pulsed UV radiation. In some embodiments, described below with reference to FIGS. 6A-6C and 7, the UV radiation is applied in concert with visible and/or infrared (IR) radiation, so as to combine the effects of UV treatment with photothermolysis. Light guide 22 concentrates the radiation on a small, well-defined area of the skin, so as to provide the high energy flux required for effective treatment of conditions such as psoriasis, while avoiding unwanted irradiation of healthy skin areas. System 10 may also be used to treat other skin conditions, such as vitiligo.

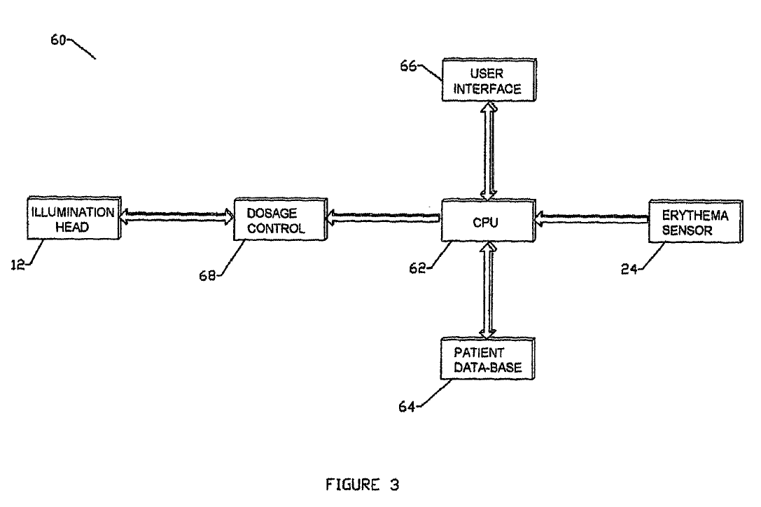

Camera unit 24 serves as an erytherna sensor, typically comprising a high-resolution color video imaging device, used to monitor the treated area of the skin. Unit 24 preferably comprises a zoom lens 26, enabling the sensor to be aimed and focused properly on the treatment area. Preferably, unit 24 also includes integral illumination sources 28, most preferably based on a light emitting diode (LED) matrix or photoluminescent lamps, emitting white light. Console 16 monitors the redness and, optionally, other parameters provided by unit 24 in order to control the duration and intensity of the treatment. Details of these monitoring and control functions are described below with reference to FIG. 3.

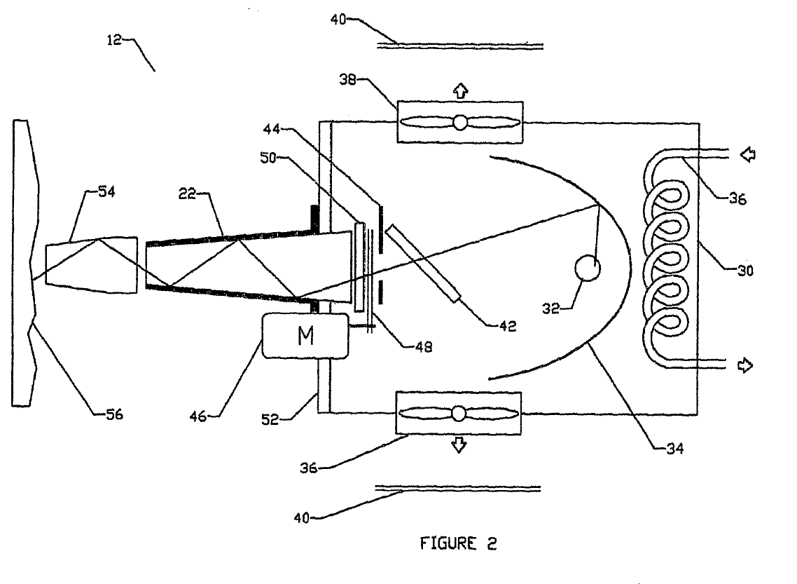

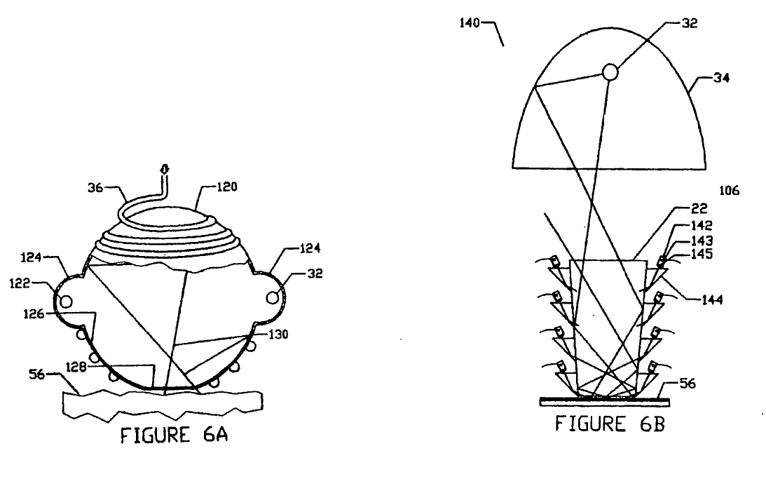

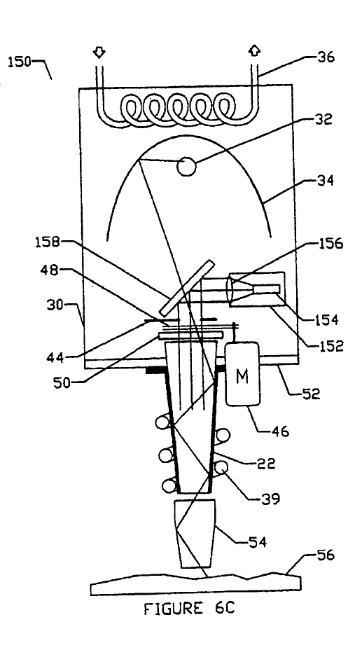

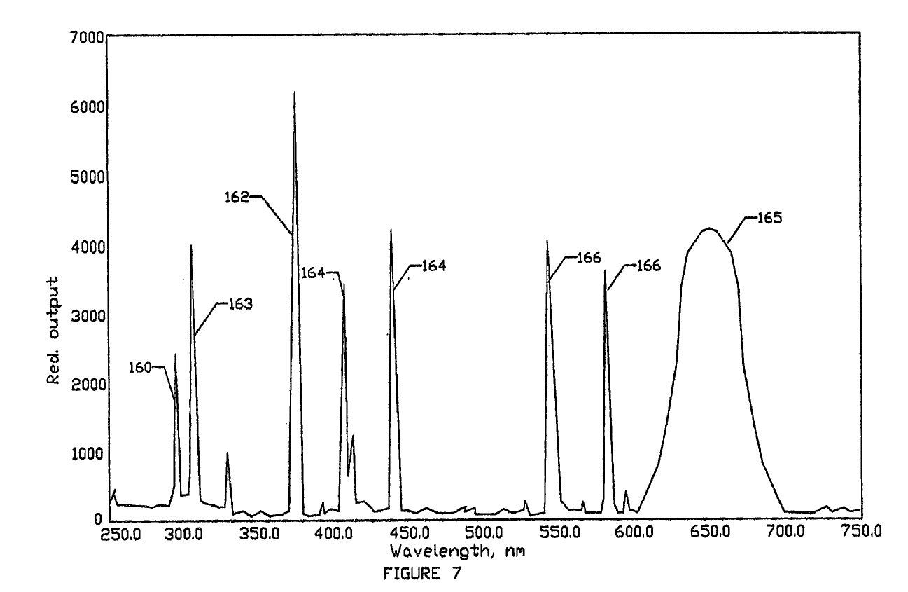

FIG. 2 is a schematic, internal view of optical head 12, in accordance with a one embodiment of the present invention. An enclosure 30 contains a lamp 32 with an elliptical reflector 34. These elements are shown in greater detail in FIGS. 5A and 5B below. Lamp 32 preferably comprises a metal-halide lamp short arc high pressure or medium pressure lamp, containing mercury and possible other additives, such as bismuth, aluminum and iron. When integrated as a metal-halide additive to a metal halide lamp, aluminum has spectral emission lines at 308 nm and 309 nm, while bismuth has an emission line at 306 nm. Mercury has emission lines in the relevant UVB treatment spectrum of 296 nm, 303 μm, and 313 nm. The mercury content in a typical 400 watt metal halide lamp used in one embodiment of the invention is in the range of 30-50 mg. In another embodiment, bismuth-halides are added to the lamp volume in a typical weight of 0.1-0.5 mg and aluminum-halides of weight 0.1-0.5 mg. Iron may be added to further enhance the UVA spectral band output of the gas discharge lamp. Alternatively, other types of lamps may be used, such as excimer lamps, as detailed in the figures and description that follow. As shown in FIG. 6C below, the lamp and enclosure may be cooled by a coolant flowing through a heat-exchanger coil 36, along with optional exhaust fans 38 (which also remove ozone that may accumulate in the enclosure). Light baffles 40 prevent the escape of stray UV light through fans 38.

Reflector 34 focuses radiation emitted by lamp 32 into light guide 22. An optional hot mirror 42 (i.e., a short-pass filter) cuts off long-wavelength radiation emitted by the lamp and reflects this radiation towards fans 38. An iris 44 is adjustable in size to control the intensity of radiation that reaches the light guide. A motor 46 (or alternatively, a solenoid) opens and closes a shutter 48 to control the duration of treatment. A UV bandpass filter 50 selects the range of wavelengths that will be conveyed from lamp 32 to the patient's skin. Preferably, filter 50 eliminates wavelengths below 300 nm, which are known to cause erytherna while providing little therapeutic benefit for psoriasis. Due to the high angular and thermal sensitivity of narrowband interference filters as to their central wavelength for spectral selection, as well as their typical low peak-wavelength transmission (15-30% typical), as well as for economical reasons due to their high cost, absorption filters may be preferred for this purpose. The absorption filters have economical prices and are limited in their spectral bandwidth selection capabilities in the UV spectrum. Typically, such filters have a pass band in the range of 100 nm to 30 nm minimum spectral bandwidth.

In one preferred embodiment a WG305 bandpass filter, made by Schott (Germany) is used to filter the required UV spectrum. In another embodiment a combination of two filters is used, installed in series, such as the Schott WG305 and UG11 filters. This combination is required to cut off more energy in the more hazardous UV spectrum under 300 nm. Optionally, filter 50 is adjustable to select the optimal UVB wavelength band based on characteristics of the patient's skin. The exposure duration of the affected skin area, controlled by shutter 48, is also based on the characteristics of the patient's skin. These characteristics may be determined by sensor 24 (FIG. 1) and/or may be input by the operator of system 10. The optical elements in head 12 are preferably mounted on an optical bend 52, which is built into enclosure 30.

Light guide 22 comprises a tapered hollow prism or conical structure, whose inner surface is coated to provide efficient reflection of the UVB radiation emitted by lamp 32. Optionally, the hollow structure contains a UV-transmitting liquid. Alternatively, the light guide may comprise a solid structure, made of suitable UV-transparent material, such as quartz or sapphire. Structures that combine hollow and solid sections may also be used. The light guide is tapered to provide enhanced concentration of the therapeutic radiation on the skin. Preferably, the light guide terminates in a removable hood 54, which is brought into contact with a surface 56 of the patient's skin. Further preferably, a gel is applied to surface 56 before bringing hood 54 into contact therewith, in order to cool the skin, as well as to improve optical index matching between the light guide and the skin surface. Because hood 54 is far removed from lamp 32, it typically remains at or near room temperature and does not itself contribute to heating of the skin. Alternatively hood 54 is further cooled to close to zero degrees Celsius to provide enhanced cooling of the irradiated skin area, in order to remove excess heat from the irradiated skin, thus avoiding or reducing thermal damage.

FIG. 3 is a block diagram that schematically illustrates dosage control circuitry 60 used in system 10, in accordance with another embodiment of the present invention. A central processing unit (CPU) 62, typically contained in console 16, receives data input from the erytherna sensor. As noted above, the sensor preferably comprises high-resolution digital imaging device 24, but it may, additionally or alternatively, comprise a dedicated composite color analyzer and/or skin texture sensor. Such a sensor may be integrated into the imaging module. The images output by the sensor are processed using image processing techniques known in the art to determine the redness of the skin and other features indicative of the effect of the treatment and the level of erythema created on the treated skin. Still further alternatively, the sensor may be a separate unit (not shown in FIG. 1) that is operated off-line and is attached by a cable to system 10.

The erythema sensor may be used either for on-line sensing, giving feedback during treatment with system 10, or for intermittent, off-line evaluation, or for both. In the off-line evaluation mode, erythema testing is used to define the exposure dose at the beginning of the course of treatment, by exposing the patient's skin to various dosage levels and testing the skin reaction after 24-48 hours to determine what dose corresponded to the erythema level threshold. The results of both this off-line evaluation and of on-line monitoring are stored in a patient database 64 for subsequent reference by CPU 62 and by the operator of system 10. Before and after each treatment, sensor 24 records the condition of the skin in the database. The doctor administering the treatment is thus able to see how the local skin condition changes from one treatment to another via a user interface 66.

Based on input from the erythema sensor and from database 64, CPU 62 operates a dosage control output 68 to regulate the functions of optical head 12. These functions typically include the radiation intensity (preferably controlled by the aperture size of iris 44), the exposure duration and, optionally the range of exposure wavelengths. In addition to automatic dosage control by CPU, the doctor administering the treatment can manually control the dosage parameters via user interface 66.

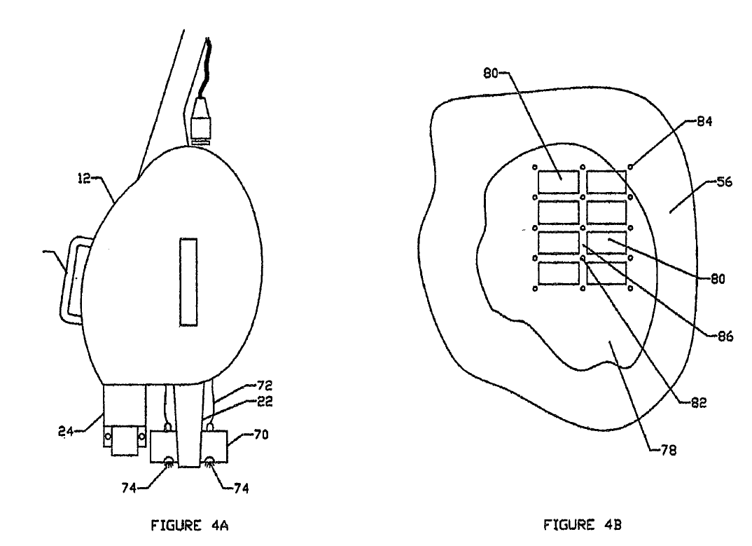

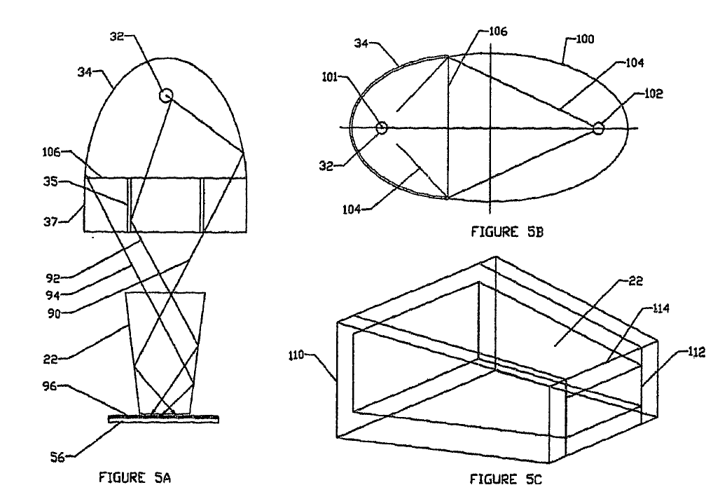

FIG. 4A is a side view of optical head 12 showing details of a marking device 70 used in automated registration of the treated area of skin 56, in accordance with another embodiment of the present invention. Device 70 is mounted on light guide 22 (or specifically on hood 54, shown in FIG. 2) and is connected by a cable 72 to CPU 62 via optical head 12. In each position at which the light guide administers radiation to skin 56, CPU 62 controls a set of markers 74 to mark the periphery of the treated area. Device 70 thus allows accurate registration and tracking of the area that has been treated, even if the patient moves during treatment. In one embodiment, device 70 comprises an ink jet module, and markers 74 comprise ink ports, preferably three or four such ports.

In another embodiment, the gel applied to skin 56 before administration of the phototherapy comprises a dyed photopolymer. Markers 74 comprise laser diodes or LEDs, which activate the photopolymer, causing it to change color. Alternatively, the photopolymer may be UV-activated, so that the radiation applied by light guide 22 causes the color change, typically after 1-2 sec of irradiation.

FIG. 4B is a schematic top view of a region of skin 56 that has been treated using system 10 and marked by means of marking device 70. In this example, a psoriatic plaque 78 has been treated by application of UV radiation to a grid of rectangular treatment areas 80, defined by the rectangular shape of light guide 22. Each area 80 is marked by markers 74 with three to four spots 82, 84 at its corners. Each spot 82 that is inside the treatment region receives radiation from all four of the surrounding treatment areas 80. Although these spots are outside the actual area contacted by the light guide, the gradual drop-off of radiation around the edges of the light guide ensures that these internal spots 82 will receive sufficient therapeutic radiation. Similarly, border areas 86 within the treatment region receive sufficient radiation from adjoining treatment areas 80.

Spots 84 mark the outer extreme of the treatment region, which preferably corresponds to the edge of plaque 78. The UV radiation typically drops off sharply at the spots and beyond, so that normal, healthy skin is not substantially affected by the UV radiation. Thus, by observing and controlling the pattern of spots 82, 84, the operator of system 10 can ensure that pathological regions of the skin receive an accurate, intense, substantially uniform dose of radiation, while preventing undesired exposure of the remaining skin.

In another embodiment, the image captured by camera unit 24 of plaque 78 and its edge contours, including spots 82, 84 and the matrix of treatment areas 80, is analyzed in real time by CPU 62 in system 10. A set of electromechanical actuators and/or motors that is integrated into adjustable suspension arm 18 is activated by the CPU through D/A drivers (not shown) using the analyzed image content data. In this way, the CPU automatically moves treatment head 12 from one treatment position to the next, in order to optimally cover the entire affected area. Spots 82, 84 serve as guide marks for the CPU.

In another embodiment of the invention, the area of the skin 56 surrounding and in proximity to the perimeter of the affected skin area, is coated by a UV radiation absorption protection layer, prior to each of the treatment procedure sessions. The material is preferably based on a fast-curing gel or cream containing additive material or dyes that have high absorption and/or reflection to UV radiation. Such materials include titanium oxide and barium titanate. The material is applied by a brush or any other appropriate mechanical means and is used for masking the non-affected skin area in close proximity, typically within 3-5 cm, around the perimeter of the affected area. The masking material after curing is of solid yet flexible texture, similar to peeling mask material used for skin treatment in cosmetic procedures, and can be easily peeled off by pulling the material layer off the coated area, in a simple procedure done by the operator of system 10.

FIG. 5A is an optical ray trace diagram showing details of the operation of reflector 34 and light guide 22, in accordance with a another embodiment of the present invention. As shown by rays 90 and 94, the arrangement of elliptical reflector 34 and tapered light guide 22 provides highly-efficient collection of the radiation emitted by lamp 32. Ray 94 is the ray characterizing the maximum acceptance angle of light-guide 22, relative to the bundle of collected rays emerging from reflector 34. Optionally, reflector 34 contains a number of sub-reflector units or flaps 35, preferably square in shape and perpendicular to the plane of an exit aperture 106 of the reflector, held by a frame 37 fitted to the aperture. The flaps are designed to reduce the output numerical aperture of the emerging light ray bundle from reflector 34 by converting part of the diverging rays, which would otherwise be lost, back to the acceptance angle range of light-guide 22. Thus, rays such as ray 92 are able to enter the aperture of light-guide 22 at an angle that will enable the entering ray to emerge at the exit aperture of the light-guide. As mentioned above, a layer of gel 96 is preferably applied to skin 56, in order to improve the optical index matching between the end of light guide 22 and the skin.

FIG. 5B is a schematic optical diagram showing details of reflector 34, which corresponds to a portion of an ellipse 100. Lamp 32 is mounted at a first focus 101 of ellipse 100, so that radiation emitted by the lamp is concentrated at a second focus 102. For efficient collection of the energy from lamp 32, this second focus 102 is preferably at or near the upper surface of light guide 22. Extreme rays 104 emitted from lamp 32 define the numerical aperture, and hence the efficiency, of the optical system. The angle of rays 104 is, in turn, determined by the geometrical properties of ellipse 100 and of mirror 34, including the major and minor axes of the ellipse and a clear aperture 106 defined by the cut of the mirror.

As lamp 32 has an elongated shape and is not a perfect point source in some embodiments, due to the nature and the shape of the glowing plasma of an arc light source, reflector 34 may alternatively have a semi-elliptical or other non-symmetric cross-section, so as to more efficiently collect the radiation emitted by the lamp.

FIG. 5C is a schematic, pictorial illustration showing one possible configuration of light guide 22, in accordance with another embodiment of the present invention. As noted above, the light guide is preferably tapered from a larger entry height 110, at which the rays from lamp 32 enter the light guide, to a smaller exit height 112. In the pictured embodiment, the exit aperture of the light guide has a larger width 114 than its height 112, in order to provide energy concentration, generating a larger energy flux, and to create the convenient treatment area coverage of the rectangular irradiation profile shown, for example, in FIG. 4B. Typically, height 112 is about 20 mm, while width 114 is about 40 mm.

In other embodiments, different sizes and height/width ratios may be used. Light guide 22 may alternatively be shaped to transform the input beam from a square or rectangular profile to an elliptical or circular profile, or vice versa. The output beam profile of the light guide can thus be selected in order to meet the specific treatment requirements in each case. Optionally, different light guides can be interchanged by the operator of system 10, either by replacing the entire light guide or replacing only hood 54 (FIG. 2).

FIG. 6A is schematic, pictorial illustration of an optical head 120 used to apply dual-band phototherapy to skin 56, in accordance with one embodiment of the present invention. Head 120 is shown in a partial cutaway view, so that both the inside and outside of the head are visible. The optical head comprises UV lamp 32, as described above, along with a lamp 122 that emits in the red range, preferably between 580 and 635 nm, which is effective in causing photothermolysis of blood vessels in the psoriatic tissue under treatment. Psoriasis-affected epidermal tissue has an underlying network of blood vessels, notably capillaries, that absorb incident radiation. Because of their small diameter, these vessels have short thermal relaxation times, and short exposure times are therefore preferable for use in photothermolyzing them. Preferably, lamp 122 comprises a gas discharge lamp, such as a xenon lamp, which most preferably operates in a pulsed mode, emitting pulses that are typically of 50-500 ms in duration. Alternatively, lamp 122 may be replaced by a high-power LED array or by a suitable laser source.

As noted above, by combining lamps 32 and 122 in a single optical head, system 10 is able to administer simultaneous UV phototherapy to psoriatic plaques and photothermolysis to coagulate the vasculature associated with the plaques. Both UV phototherapy and photothermolysis are threshold-type effects, meaning that the radiation flux incident on the plaque must be above a certain minimum level in order to achieve useful therapeutic results. On the other hand, increasing the incident flux in either of these therapies has undesirable side effects, including erythema (in the case of UV) and thermal damage to surrounding skin and underlying tissue layers (for intense visible/IR exposure). The effects of UV and visible/IR irradiation on psoriatic plaques are biologically independent of one another, with UV irradiation affecting mainly the cell structure of the outermost skin layers, while photothermolysis operates on the blood vessels deeper within the tissue. In terms of therapeutic benefit, the two types of radiation are complementary. Therefore, by combining lamps 32 and 122 in a single unit, it is possible to achieve enhanced therapeutic effect and to lower the therapeutic thresholds of both types of irradiation, without increasing the incidence of erythema and thermal damage.

In the embodiment shown in FIG. 6A, each of lamps 32 and 122 is mounted at the focus of a respective concave reflector 124, preferably a parabolic reflector. The remainder of head 120 is spherical in shape and has an internal coating 126 that diffusely reflects incident radiation over a wide range of wavelengths, from UVB through near IR. Head 120 thus acts as an integrating sphere, with an output window 128 that is placed against skin 56. Rays 130 emitted by both of lamps 32 and 122 are reflected diffusely from the interior wall of the sphere until they impinge on skin 56 through window 128.

FIG. 6B is a schematic side view of another optical head 140 for use in dual-band phototherapy, in accordance with another embodiment of the present invention. In this embodiment, UV lamp 32 is mounted at the focus of elliptical reflector 34, which focuses the UV radiation into light guide 22, as described above. Visible radiation for photothermolysis is provided by an array of high-power IR emitting modules 142, based on LEDs or diode lasers. Each of the modules includes collimating optics 145 and a semiconductor chip 143. The modules are mounted against respective prisms 144. These prisms are fixed, typically by optical cement, to the sides of light guide 22, so as to inject the visible light emitted by modules 142 into the light guide. The combined UV and visible beams are then incident on skin 56.

FIG. 6C is a schematic side view of still another optical head 150 for use in dual-band phototherapy, in accordance with another embodiment of the present invention. The arrangement of head 150 is similar to that of head 12, shown in FIG. 2, and only the differences in the present embodiment are described here. Head 150 includes a visible light source 152, based on a pulsed lamp 154, such as a gas discharge lamp or on a LED or diode laser array, in addition to UV lamp 32. Alternatively, lamp 154 may be replaced by a suitable laser. Light from lamp 154 is collimated by a lens 156 and is reflected into light guide 22 by a dichroic reflector 158, which reflects visible light while passing UV radiation emitted by UV lamp 32. Cooling coils 39 are preferably applied to light guide 22, while cooling coil 36 cools housing 30. Cooling coil 39 can alternatively comprise Peltier effect-based cooling elements, both alternatives having the function of keeping the light guide at a temperature much under room temperature, preferably close to zero temperature, in order to extract from and dispose of the excess heat generated by the combined UV and visible/IR radiation at the irradiated tissue.

FIG. 7 is a spectral plot of radiation generated by a dual-band optical source for performing combined UV and visible treatment of psoriatic plaques, in accordance with yet another embodiment of the present invention. As noted above, lamp 32 preferably comprises a metal-halide lamp, containing mercury, as is known in the art. The lamp may contain other metal additives, as well, such as tantalum, vanadium and/or cesium, as well as halogens, in order to provide visible radiation that has useful photothermolytic effect. In this case, as long as the visible radiation is sufficiently strong, it may be possible to carry out the combined therapy without a separate visible light source, in distinction to the embodiments of FIGS. 6A-6C.

Thus, in the spectrum shown in FIG. 7, lines 160, 162 and 164 are characteristic mercury-halide spectral features. Lines 160 are the two UVB spectral lines that are commonly used using fluorescent light sources for treating psoriatic plaques by DNA destruction in the 300-320 nm range. Line 162 is a UVA line, while lines 164 are violet lines that are thought to provide useful secondary healing effects, operating on the body's auto-immune system. Lines 166 in the yellow/orange range, emitted due to the optional metal additives mentioned above, have photocoagulative (photothermolytic) effects on the small blood vessels that nourish the affected tissues.

In addition to these lines (or instead of lines 166), a broad band 168 is generated by an additional light source provided for the purpose of photothermolysis, such as lamps 122, 142 or 152, shown in FIGS. 6A-6C. Alternatively, other spectral distributions may be used. Furthermore, although the use of this combination of UV and visible radiation bands is described herein mainly with reference to psoriasis, those skilled in the art will appreciated that the combined effects of UV phototherapy with visible-light photothermolysis may also have therapeutic benefit in treatment of other skin disorders.

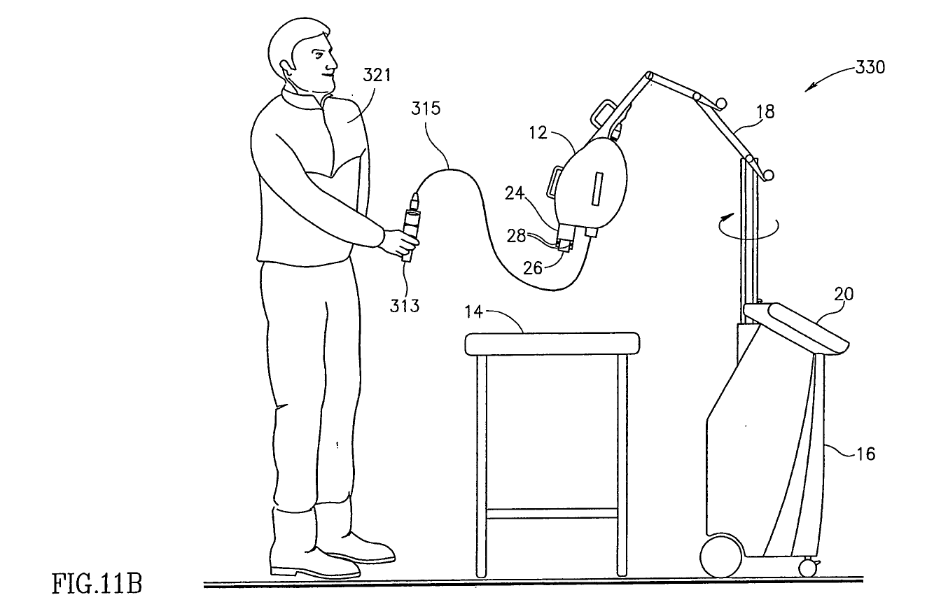

FIG. 8 is a spectral plot of typical radiation generated by an excimer lamp-based light source for performing narrow band UVB, or combined UV and visible treatment of psoriatic plaques, in accordance with yet another embodiment of the present invention. In the spectrum shown in FIG. 8, line 180 is characteristic of XeCl excimer radiation. It is similar to the narrow UVB spectral line that is produced by excimer laser light sources for treating psoriatic plaques by DNA destruction in the 300-320 nm range, but has a broader spectral width. The typical excimer laser bandwidth is 0.3±0.2 nm while the typical spectral bandwidth of an excimer lamp output is 2 nm. The main benefits of the excimer lamp light source in comparison to the excimer laser are continuous-wave operation (CW), non-coherence, compact size, spectral output, tunability by driving frequency, higher reliability and much lower costs.

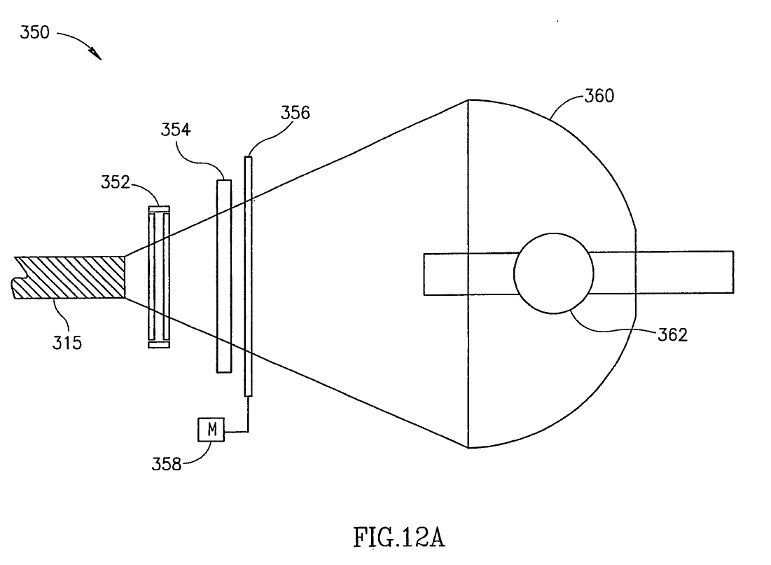

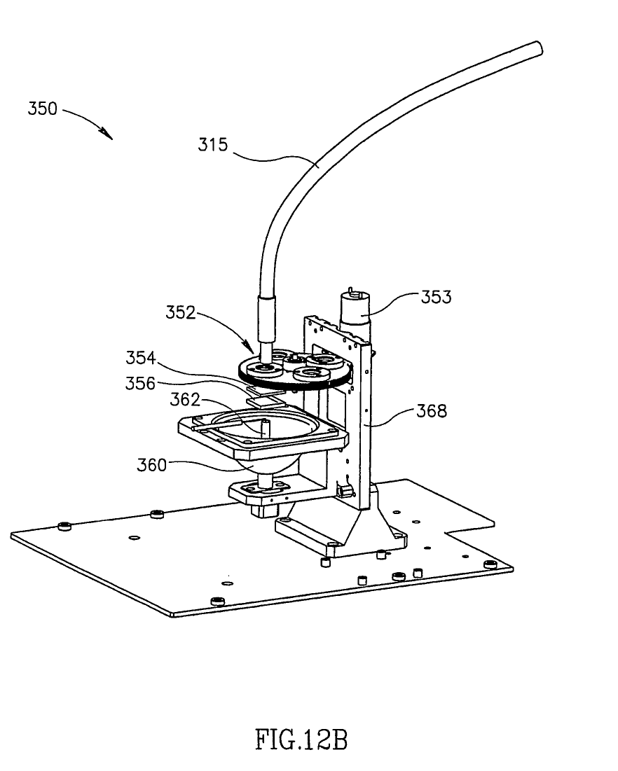

FIG. 9A is a typical cross-sectional image of an excimer lamp 200 used as a light source in skin treatment apparatus according to one embodiment of the invention. The lamp is constructed of two concentric glass tubes 214 and 208, wherein an excimer gas is inserted and trapped in a space 212 between the two tubes. When a high frequency (RF) alternating electric field is induced by a power source 218 between two electrodes 210 and 216, the gas in the volume between the two tubes starts to glow. UV light 221 emerges between the external elements of electrode 210. Cooling liquid is circulated in a volume 220 of inner tube 214 in order to enable stable light output and operation of the light source.

FIG. 9B is a cross-sectional image of another excimer lamp 240 used as a light source in skin treatment apparatus according to another embodiment of the invention. The lamp is constructed of two concentric glass tubes 255 and 256, preferably quartz, wherein an excimer gas is inserted and trapped in a space 250 between the two tubes. A light-reflective electrode 253 is coated on the external surface perimeter of inner tube 255, and a second electrode 254 is coated on the internal surface perimeter of outer tube 256. When a high frequency (RF) alternating electric field is induced between the two electrodes 253 and 254, the gas in the volume between the two tubes starts to glow and light 258 is trapped and directed out of space 250 by multiple reflections between the two reflective electrodes 253 and 254. Part of the generated light that is not reflected by electrodes 253 and 254 is trapped in the glass walls of tubes 255 and 256 and is directed out by multiple internal reflections within the walls, acting as light guides.

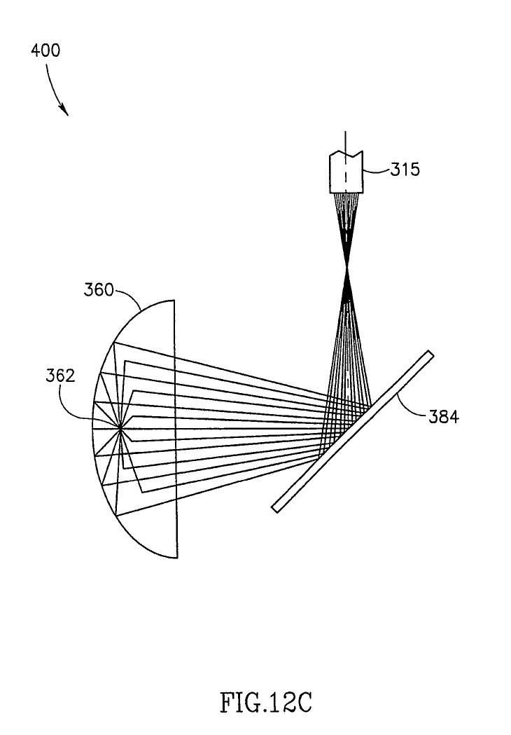

The light emerging from lamp 240 has an annular or ring shape 261, with a low- to zero-energy area in its center 260. The ring-shaped output energy beam emerges through the entire aperture created by the volume between the two tubes 255 and 256. Cooling liquid is circulated inside a volume 252 of inner tube 255, in order to enable stable light output and operation of the light source. The small-diameter circular beam of lamp 240 enables optimal collection of the generated light into light-guides of various types, like light guide 15 shown in FIG. 1B and the light-guide integrated to the treatment head shown below in FIG. 10.

FIG. 10 is a schematic side view of still another optical head 300 for use in UVB or in dual-band phototherapy, in accordance with another embodiment of the present invention. The arrangement of head 300 is similar to that of head 12, shown in FIG. 2, and only differences in the present embodiment are described here. Head 300 includes a UVB light source 310, based on the excimer lamp embodiment shown in FIG. 9B. Light from source 310 is collimated by a lens module 314 and is further directed in a small divergence angle into light guide 22. Cooling coils 318 are used to cool the internal volume of the inner tube of lamp 310. Pump 320 circulates the cooling liquid in cooling coils 318. Cooling unit 318 and pump 320 are part of a dedicated liquid cooling module 315.

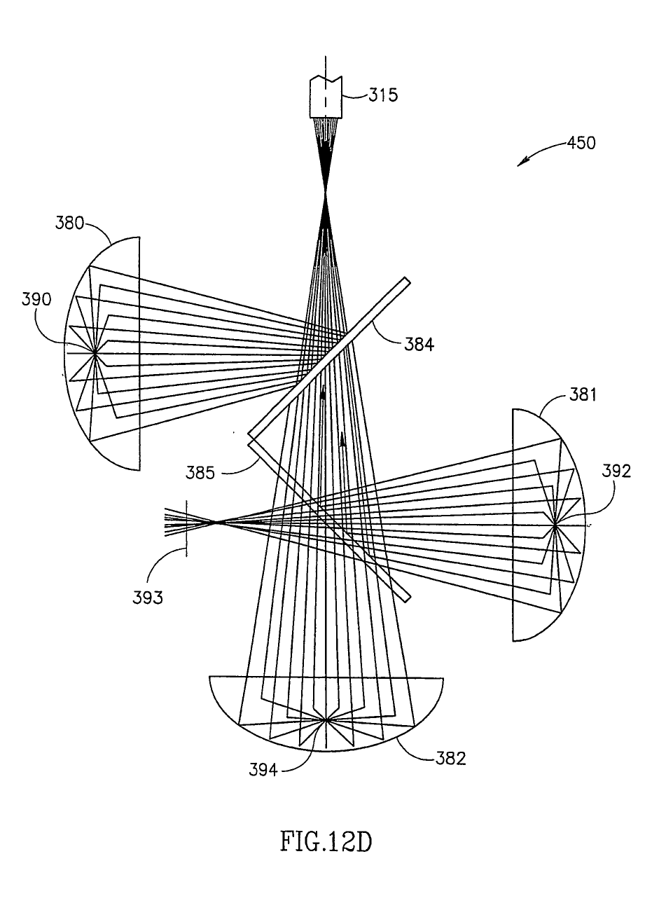

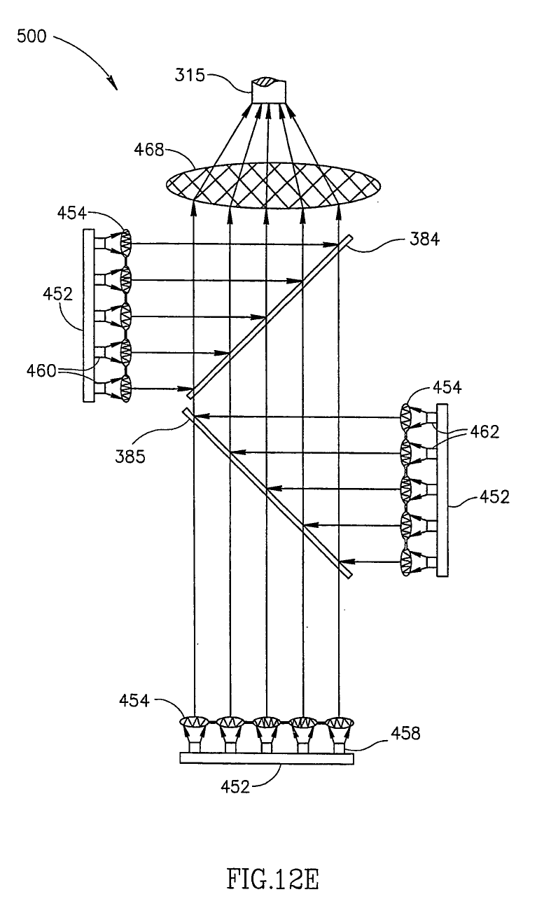

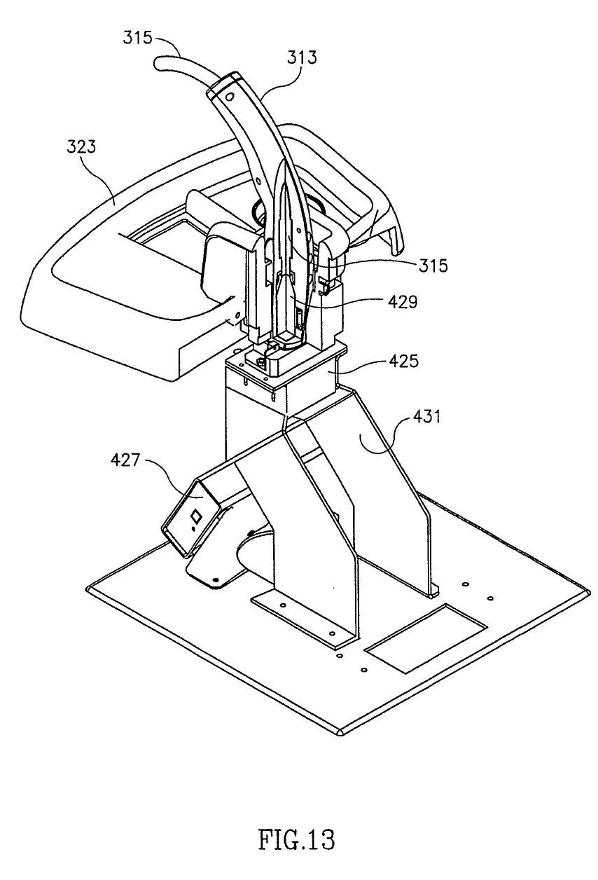

FIG. 11A is a schematic, pictorial illustration of a system 311 for phototherapeutic treatment of psoriasis and other skin disorders, in accordance with another embodiment of the present invention. System 311 comprises an optical head 313, which is used to collect and concentrate the light and irradiate the skin of a patient (not shown), who typically lies on treatment table 14. Optical head 313 is coupled to and connected through a flexible fiberoptic light guide 315 with a broad spectral passband to a power supply and control console 317. Light guide 315 may comprise a fiberoptic light-conducting bundle or a liquid-filled flexible light conductor, for example Lumatec Lightguide Series 250, produced by Lumatec (Munich, Germany).

Console 317 includes a light source (not shown in this figure) coupled to the light guide. The flexibility of the light guide allows an operator 321 of system 311 to adjust the vertical, horizontal and radial placement of optical head 313 relative to the patient's body. A mechanical or electromechanical elevation element 319 enables the adjustment of the fiber optic bundle output to the level required for the specific treatment conditions. The light source is preferably capable of providing radiation to light guide 315 in multiple different wavelength bands, with automated selection of the desired bands, as described hereinbelow.

Light guide 315 is preferably capable of conveying radiation with high transmittance down to at least 290 nm. As noted above, a quartz fiber bundle may be used for this purpose. Alternatively, the light guide may comprise a tube filled with UV-transmitting liquid. This latter alternative has the advantages of high transmittance (roughly 75% over a two-meter length) and high numerical aperture (NA), typically about 0.57. (By comparison the NA of quartz is about 0.24.) Suitable light guides of this type are available from Lumatec and from Rofin (Perth, Australia), for example.

FIG. 11B is schematic, pictorial illustration of a system 330 for phototherapeutic treatment of psoriasis and other skin disorders, in accordance with another embodiment of the present invention. In this embodiment, optical head 12 is coupled to handheld treatment unit 313 using light guide 315, which has a broad spectral band. Connection between light guide 315 and optical head 12 is typically made through a quick-release, interchangeable mechanical interface. Optical head 12 includes a light source and light collection optics (not shown in this figure) coupled to the fiber bundle. The flexibility of light guide 315 provides ease in operational positioning and distancing of the light exit aperture from the light source itself.

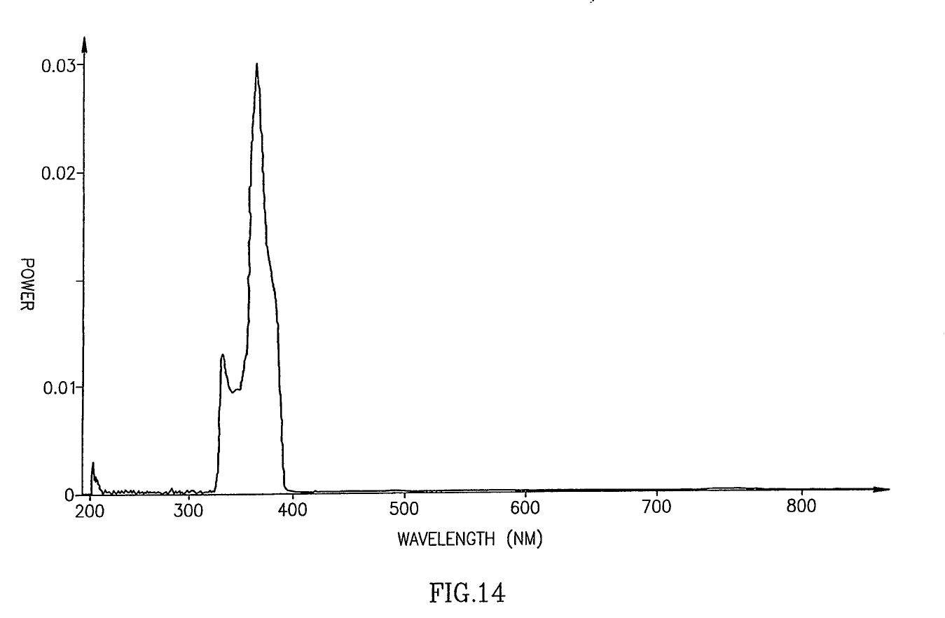

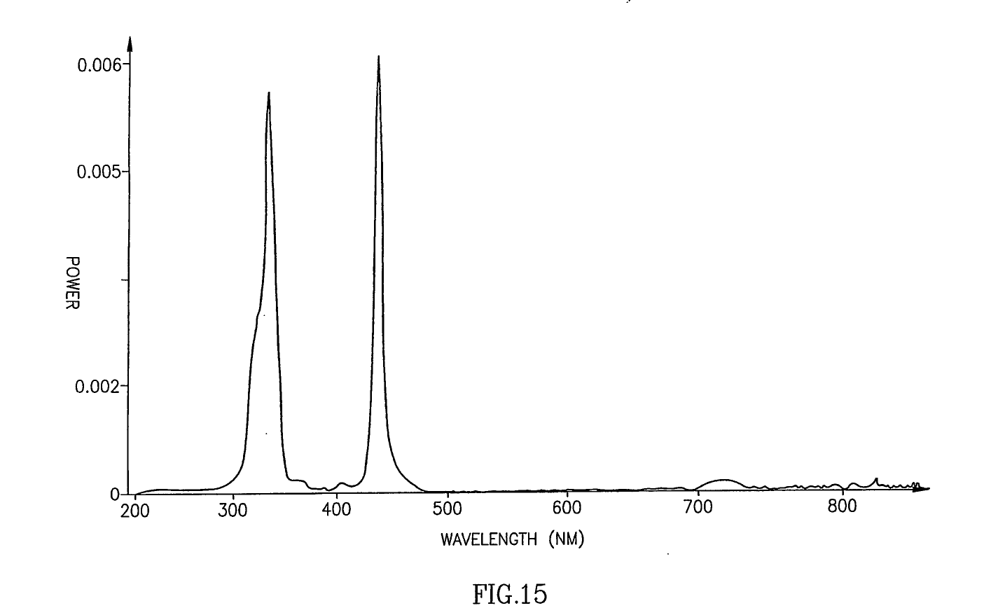

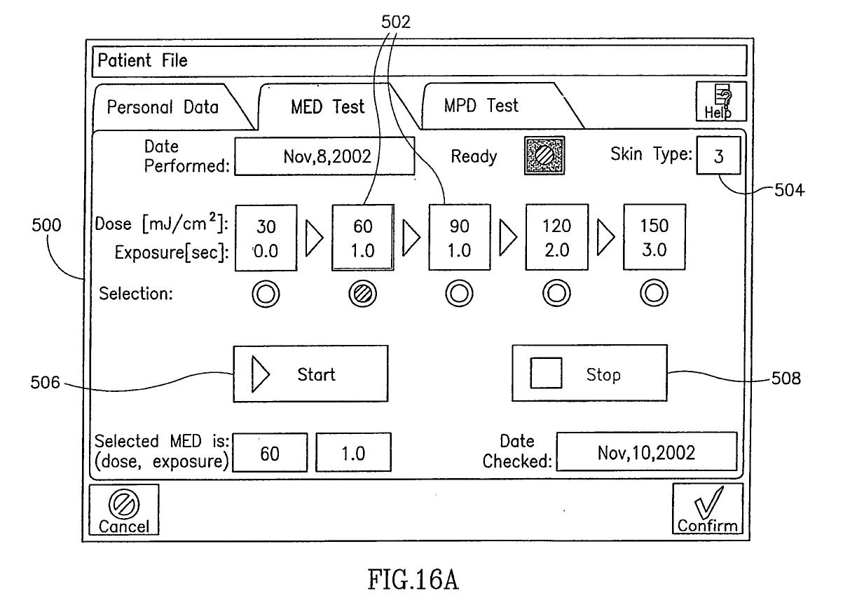

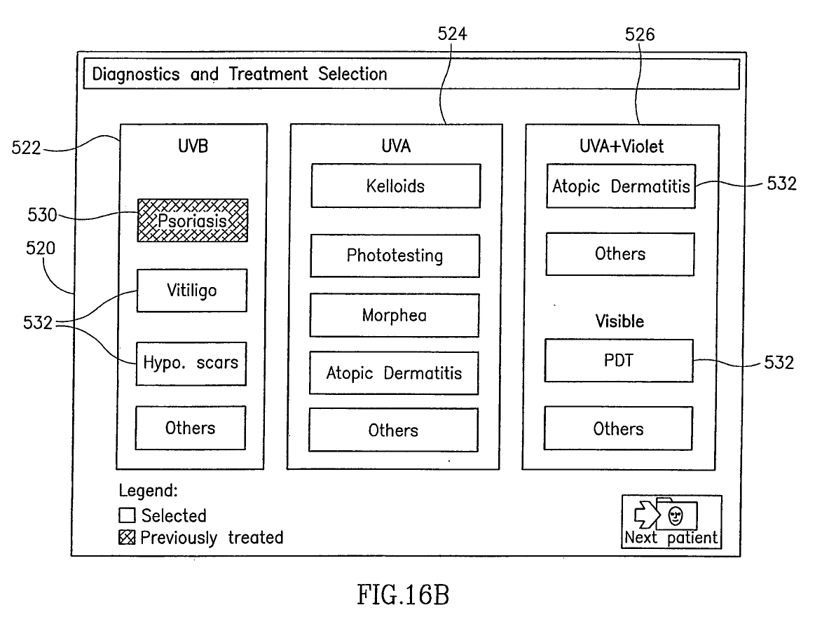

FIG. 12A is a schematic, sectional view of optics 350 for feeding radiation into light guide 315, in accordance with an embodiment of the present invention. Optics 350 may be used, for example, in console 317 (FIG. 11A). A lamp 362 emitting UV radiation is positioned at the focus of an elliptical reflector 360, which concentrates the radiation from the lamp into light guide 315. A filter 352 selects the wavelength range or ranges to be admitted to the light guide. A hot mirror 354 is provided to reflect long-wavelength radiation away from light guide 315, in order to avoid undue heating. A shutter 356 is controlled by a motor 358 in order to block and open the light path to the light guide as desired.

Preferably, lamp 362 comprises a gas discharge lamp, most preferably a high-pressure gas discharge lamp, such as a metal halide lamp. The working pressure inside such a lamp is typically in the range of 50-60 atm. Examples of such lamps include the Osram (Munich, Germany) HBO 250 lamp and the Perkin Elmer Optoelectronics (Wiesbaden, Germany) XHP200 lamp. As noted above, certain additives may be used in the lamp to enhance emission in desired spectral bands, for example: bismuth for UVB emission; cesium or iron for UVA emission; and gallium for violet light emission. A combination of such additives may be used to provide strong emission in multiple bands simultaneously. Because high-pressure discharge lamps have a very small, intense arc (typically only 1-3 mm across), the radiation from the lamp may be focused very efficiently into light guide 315. In fact, it may be desirable to place the lamp so that the arc is slightly off the focus of reflector 360, in order to avoid creating a hot spot on the light guide. Alternatively, a medium-pressure lamp (working pressure of 1-3 atm) may be used.

Filter 352 may be one of a selection of filters provided in a filter wheel. Alternatively, other electrically or electromechanically tunable optical filter types may be used, as are known in the art, such as PLZT or KDP crystals with appropriate polarizers. The choice of wavelength range or ranges may be controlled automatically by console 317, depending on the desired therapeutic modality, or manually by operator 321. Preferably, the lamp and filters are chosen so as to allow multiple different therapeutic wavelength ranges to be used simultaneously, including not only UVB, but also UVA and visible (typically violet) light. Red and/or infrared radiation for specific desired treatment requirements may be selected and provided, too, as described above. Methods for selecting and controlling the wavelength and power characteristics of the radiation delivered through light guide 315 are described in greater detail hereinbelow.

FIG. 12B is a schematic, pictorial illustration of one possible embodiment of optics 350, showing further details of their mechanical construction. The optics are mounted on a base 368. A mechanical assembly 353 is used to select filter 352 by rotating the filter wheel in which the filter is held.

FIG. 12C is a schematic, sectional view of optics 400 for feeding radiation into light guide 315, in accordance with another embodiment of the present invention. In this case, the optical axis of reflector 360 is turned 90° by a cold mirror 384, which reflects only short-wavelength radiation. The wavelength band within which the cold mirror reflects radiation may be chosen to give a sharp cut-on and cut-off, in order to select the band of radiation to be conveyed by the light guide. Radiation outside this band passes through the cold mirror and is discarded.

Reflector 360 in this embodiment may be chosen to have a longer focal length than that in the embodiment of FIG. 12A. The longer focal length gives a longer focal path from lamp 362 to light guide 315, and thus allows a light guide with lower NA to be used without substantial radiation loss at the entrance to the light guide.

FIG. 12D is a schematic, sectional view of optics 450 for feeding radiation to light guide 315, in accordance with a further embodiment of the present invention. Optics 450 comprise three lamps 390, 392 and 394, which emit radiation in different, respective wavelength bands. Each of the lamps is provided with an elliptical reflector 380, 381 or 382, all of which are focused on the entrance to light guide 315. Typically, lamp 390 operates in the shortest wavelength range, with lamps 392 and 394 operating at successively longer wavelengths. Alternatively, larger or smaller numbers of lamps may be used, with different wavelength arrangements. Dichroic reflectors (beam-splitters) 384 and 385 are used to combine the beams from the different lamps, substantially without loss. The undesired light bands that may be emitted by each lamp are transferred through the dichroic reflectors, such as dichroic reflector 385. This non-useful light energy is concentrated at a focal point 393, where a heat exchanger (not shown) may be placed in order to absorb and further discharge the heat generated by this ineffective energy. Lamps 390, 392 and 394 are switched on and off in order to give the desired wavelength band, or any combination of bands, in light guide 315.

FIG. 12E is a schematic, sectional view of optics 500 for feeding radiation to light guide 315, in accordance with an alternative embodiment of the present invention. This embodiment is similar in principle to that of FIG. 12D, except that solid-state light sources 458, 460 and 462 are used instead of arc lamps. The light sources may comprise, for example, laser diodes or LEDs, operating in different, respective wavelength ranges. (Laser diodes and LEDs with wavelengths down to 290 nm and power levels in the tens of milliwatts are currently available from companies such as Nichia (Tokyo, Japan), and it is expected that higher-power devices will be developed and become commercially available.) To provide sufficient optical power, the solid-state light sources may be arranged in matrices on respective substrates 452. The light sources may be fitted with lenses 454 or lenslet arrays in order to provide better collimation of their output. An objective 468 focuses the radiation into light guide 315. This arrangement is capable of providing several hundred milliwatts of UVB radiation at the input to the light guide and higher energy levels, in the range of several watts, in the UVA and at selectable visible spectral bands.

As in the preceding embodiment, dichroic mirrors or prisms are used to combine the beams from the different light sources. The light sources are switched on and off in order to select the wavelength range or ranges to be used in treating the patient.

Alternatively, solid-state light sources and one or more gas discharge lamps may be combined in a single system to provide multi-wavelength input to light guide 315. The radiation from the different types of light sources may be combined by dichroic beamsplitters 384 and 385, or a hybrid optical arrangement of the type shown in FIG. 6B may be used.

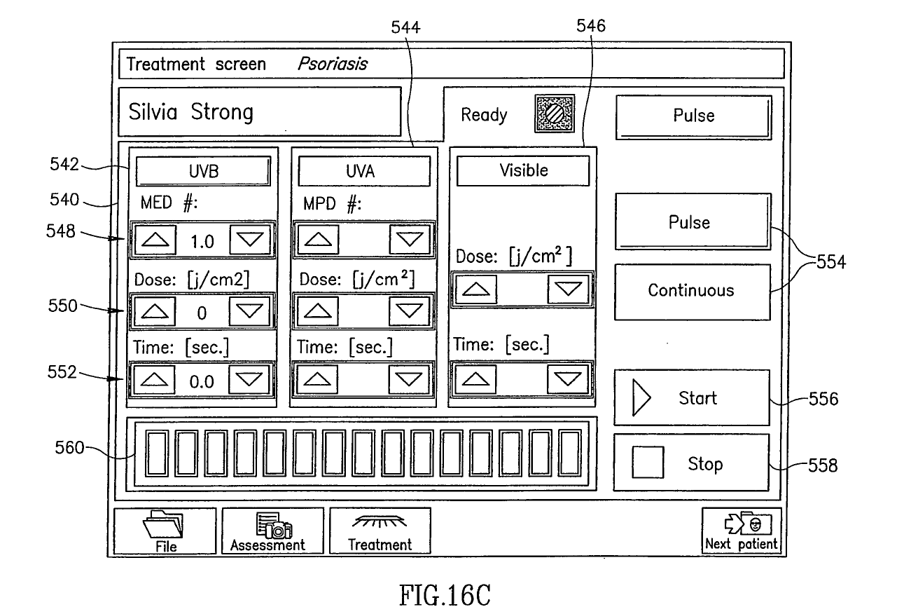

FIG. 13 is a schematic, pictorial, partly cutaway view of a receptacle 510 for receiving optical head 313, in accordance with an embodiment of the present invention. Receptacle 510 is typically integrated in console 317, alongside a tray 323 used by operator 321 during treatment. In between treatments, optical head 313 is inserted into receptacle 510. The optical head is constructed so that the end of light guide 315 engages a solid lightguide quartz prism 429. (This is the end of the light guide that is normally applied to the patient's skin.) The light source in console 317 is operated intermittently while the optical head is in the receptacle, in order to transmit radiation through light guide 315. Radiation is thus emitted from the optical head, and is conveyed by prism 429 to a detector 425, typically a broadband thermal detector.

A controller 427 is coupled to measure the signal that is output by detector 425, in order to determine the output level of the light guide in one or more of the wavelength bands provided by the system. The controller is thus able to calibrate the power output of the system and verify that the power is within the expected range. This calibration energy output data may be used in subsequent treatments to increase or decrease the power output of the radiation source in the console or, alternatively or additionally, to increase or decrease the duration of treatment provided, so that the patient receives precisely the required radiation dosage. If the power measured by detector 425 is too far outside the expected range, controller 427 may send an error signal to operator 321, and may also block use of the system until the problem is corrected.

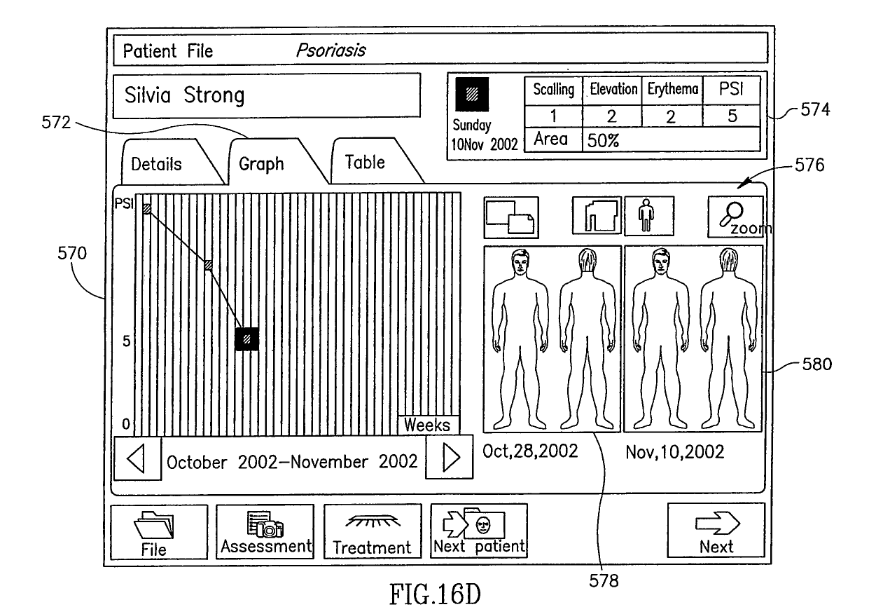

FIGS. 14 and 15 are plots that schematically illustrate spectral bands generated by system 311, for example, for use in phototherapy, in accordance with embodiments of the present invention. These spectra were obtained using a high-pressure metal halide arc lamp 362, with appropriate filters 352. In FIG. 14, a broad UVA spectral range is provided, which may be used, for example, in treating atopic dermatitis. In FIG. 15, an interference filter with dual passbands is used to convey both UVB and violet radiation. This combination may be used, for example, in simultaneously treating psoriasis (using UVB) and palliating inflammation of the skin (using violet light).

Other wavelength combinations may also be provided by system 311, for example:
UVA and violet radiation may be used together for treating atopic dermatitis and inflammation.
Violet radiation may be used in conjunction with appropriate chemical agents for photodynamic therapy (PDT).
UVA or violet radiation may be used for treatment of acne.
Red radiation, which penetrates deeper through the skin, may be used for pain relief, as in treatment of erythritis, as well as joint pain.
   Therapeutic uses of violet light are described, for example, in U.S. patent application Ser. No. 10/098,592, which is assigned to the assignee of the present patent application, and whose disclosure is incorporated herein by reference. Other applications of the multi-wavelength capabilities of system 311 will be apparent to those skilled in the art.

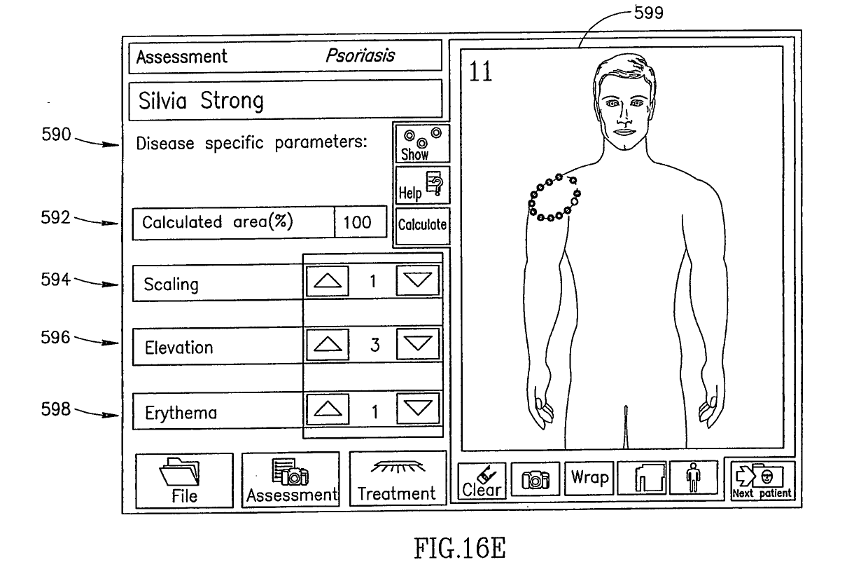

FIGS. 16A-16E are schematic representations of computer screens used in a graphical user interface of system 311, in accordance with an embodiment of the present invention. System 311 preferably maintains a database of all patients who have been treated by the system. The database includes personal and health information regarding each patient, as well as records of treatment administered to date. Prior to initiating treatment, operator 321 selects the current patient from the database, or inputs patient information, if the current patient is being treated for the first time. Data entry screens (not shown) are provided for this purpose.

As noted above, the appropriate dosage of UVB radiation to administer to a patient is typically determined based on the MED (Minimal Erythema Dose) for that patient. The MED is established by system 311 based on a standard test, which is controlled by operator 321 by means of a MED testing screen 500, shown in FIG. 16A. The operator determines the radiation dosage and exposure time to apply by selecting an on-screen dosage button 502. The available dosages are suggested by system 311 based on the patient's skin type, which is input by the operator and appears in a skin type window 504. By selecting different dosage buttons, the operator can perform up to five different tests, at different dosage levels, on different skin areas. Each test is initiated by selecting a start button 506, and the procedure may be terminated by selecting a stop button 508.

The operator then observes, after 24-48 hours, the erythema that appears on the patient's skin as a result of each test, and based on this observation, records the MED for the patient in the database of system 311. This MED level is used in determining subsequent dosage. A similar procedure may be used to determine the patient's MPD (Minimum Phototoxic Dose) for UVA treatment In order to select the type of treatment to administer the patient, operator 321 next selects a diagnosis, in an indications screen 520, shown in FIG. 16B. Screen 520 offers three treatment windows: a UVB window 522, a UVA window 524 and a UV/visible window 526. Each window contains buttons 530 and 532 providing different indications. A first button type 530 refers to indications for which this patient has already been treated (psoriasis, in the present example), as recorded in the database of system 311. A second button type 532 refers to all other indications. As shown in the figure, system 311 offers the following treatment options:
UVB treatment for psoriasis, vitiligo and certain scar types.
UVA treatment for kelloids, atopic dermatitis and morphea, as well as phototesting.
Combined UVA/violet light treatment for atopic dermatitis.
Visible light application for PDT.
   Other indications may also be added, of course, either as an extension of the software options offered by system 311, or by the operator.

Based on the diagnosis, together with the patient records and treatment history stored in the database, system 311 allows operator 321 to select the dosage to be applied in this treatment, using a treatment screen 540, as shown in FIG. 16C. In the present example, in which the patient is diagnosed with psoriasis, a UVB dosage is recommended, and appears in a UVB window 542. Additional windows 544 and 546 are available for controlling UVA and visible radiation dosages. Typically, the operator selects the UVB dosage in units of MEDs, based on the MED determined for this patient, as described above. The operator may increase or decrease the number of MEDs to apply using a MED control 548. System 311 then determines automatically the intensity and duration of radiation to apply.

Alternatively, the operator may use a dose control 550 and a duration control 552 to manually increase or decrease the intensity and/or duration of the radiation dose. If a MED level is set, and the operator changes the intensity, for example, system 311 will automatically decrease the duration of exposure, and vice versa. For safety reasons, the system preferably limits the dosage to a predetermined maximum, typically 8 MEDs. The operator may also choose to apply either pulsed or continuous radiation, using mode control buttons 554.

To begin treatment, the operator presses a start button 556, or a foot pedal (not shown) attached by a cable to the system. Treatment may be terminated by pressing a stop button 558, when operating in a continuous mode. Otherwise, treatment automatically stops when the selected MED or dose has been given in pulsed mode. A bar graph 560 shows the progress of the treatment exposure duration.

As noted above, system 311 maintains a database “file” for each patient, which includes the history of treatments the patient has undergone, including digital images of the treated skin areas. The database records are updated after each treatment, or when operator 321 inputs new data. The data in the files can be read by the operator in alphanumeric, graphic and tabular forms.

FIG. 16D shows a graphic screen 570, giving the treatment history of a sample patient. The progress of the patient's condition over time is shown in a graph 572. The graph may be marked to show dates of treatment and assessment. A numerical window 574 shows numerical values of computed diagnostic data, as well. The operator may also input and store pictorial records, to track the patient's progress. In the present example, successive images 578 and 580 of this sort are displayed in a picture window 576.

FIG. 16E shows an assessment screen 590, which is used by operator 321 to record the patient's condition periodically. Various diagnostic parameters can be input and/or calculated using this screen. For example, the operator may delineate the area of a lesion on an image 599, and may calculate the area of the lesion, using an area calculation window 592. The area is typically displayed as a percentage of the original area of the given lesion, before the course of treatment began. The operator may also input scores corresponding to the redness, scaling level and elevation of the lesion, as shown in scaling and elevation windows 594 and 596. The level of erythema may be recorded in an erythema window 598. These visual inspection parameters, rated on a scale of 1 to 4, are used to calculate a Psoriasis Score Indicator (PSI), which is the sum of the three inspection parameters. These parameters allow the operator to track the patient's status over time, using screen 570, for example, and to modify treatment parameters as required.

Although the embodiments described above make reference to particular indications for phototherapy, and make use of certain types of equipment, the principles of the present invention may similarly be applied to treat other skin conditions, in other equipment configurations. It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1-7. (canceled)

8. Apparatus for treatment of a skin condition, comprising:
a radiation source, adapted to irradiate an area of the skin with radiation in at least one of an ultraviolet, visible and infrared spectral range; and
marking means, adapted to delineate the area on the skin responsive to irradiation of the area by the radiation source,
wherein the marking means comprises one or more markers, adapted to print a plurality of marks on the skin delineating the irradiated area, and
comprising an arm supporting the radiation source, and an imaging device coupled to capture an image of the skin, and a processor adapted to analyze the marks on the skin appearing in the image so as to guide the radiation source by controlling movement of the arm, responsive to the marks in the image.

9. Apparatus for treatment of a skin condition, comprising:
a radiation source, adapted to irradiate an area of the skin with radiation in at least one of an ultraviolet, visible and infrared spectral range; and
marking means, adapted to delineate the area on the skin responsive to irradiation of the area by the radiation source,
wherein the radiation source comprises a radiation guide, which is brought into proximity with the area of the skin so as to deliver the radiation thereto, and wherein the marking means is adapted to mark a periphery of the radiation guide.

10. Apparatus for treatment of a skin condition, comprising:
a radiation source, adapted to irradiate an area of the skin with radiation in at least one of an ultraviolet, visible and infrared spectral range; and
marking means, adapted to delineate the area on the skin responsive to irradiation of the area by the radiation source,
wherein the marking means comprises a photosensitive substance, which is applied to the skin prior to irradiating the area of the skin, and wherein the radiation causes a visible change in the photosensitive substance, thereby delineating the area.

11-15. (canceled)

16. A method for treatment of psoriasis, comprising:
irradiating a psoriatic area of skin with ultraviolet (UV) radiation, so as to treat the psoriasis;
detecting an optical quality of the irradiated area; and
controlling a level of the radiation responsive to the indication.

17. A method according to claim 16, and comprising, prior to irradiating the psoriatic area of the skin, applying a quickly-solidifying, self-peeling cream or gel material, having UV energy protection capability including at least one of absorption and reflection properties, to a healthy skin area on a periphery of the psoriatic area.

18-23. (canceled)

24. Apparatus for treatment of skin disorders, comprising:
a radiation source, adapted to generate ultraviolet (UV) radiation suitable for treatment of skin affected by one or more of the skin disorders; and
radiation delivery optics, coupled to the radiation source so as to concentrate and deliver the generated UV radiation directly to the affected skin with an energy flux level per single treatment and specific affected skin area selected from an energy radiation flux group consisting of at least a radiation flux equal or higher than 50 mJ/cm² in less than 30 sec per treatment and a radiation flux higher than 1.5 MED (Minimum Erythema Dose),
wherein the UV radiation is in a spectral range of 296-390 nm and comprises at least one spectral line of substantial intensity having a bandwidth of at least 1 nm, wherein the spectral line is selected so as to engender clearing of the affected skin, and
wherein the radiation delivery optics are adapted to limit an exposure area of the radiation to the affected skin.

25. Apparatus according to claim 24, wherein the radiation delivery optics comprise one of a selection of interchangeable light guides, which are adapted to direct the radiation toward the affected skin during the treatment, and
wherein the light guides are selectable so as to match an output aperture of the selected light guide to a size and shape of the affected skin.

26. Apparatus according to claim 25, wherein the output apertures of the interchangeable light guides are selected from a group including aperture areas of at least 200 mm², 400 mm² and 2500 mm².

27. Apparatus according to claim 25, wherein the radiation delivery optics further comprise aperture shaping optics at the output aperture so as to provide an aperture shape that is adapted for optimal plaque area energy coverage.

28. Apparatus according to claim 24, and comprising a module containing the radiation source and the radiation delivery optics, and an articulated arm, which is coupled to suspend and position the module relative to the affected skin.

29-30. (canceled)

31. Apparatus for treatment of skin disorders, comprising:
a radiation source, adapted to generate ultraviolet (UV) radiation suitable for treatment of skin affected by one or more of the skin disorders; and
radiation delivery optics, coupled to the radiation source so as to concentrate and deliver the generated UV radiation directly to the affected skin with an energy flux level per single treatment and specific affected skin area selected from an energy radiation flux group consisting of at least a radiation flux equal or higher than 50 mJ/cm² in less than 30 sec per treatment and a radiation flux higher than 1.5 MED (Minimum Erythema Dose),
wherein the UV radiation is in a spectral range of 296-390 nm and comprises at least one spectral line of substantial intensity having a bandwidth of at least 1 nm, wherein the spectral line is selected so as to engender clearing of the affected skin, and
wherein the radiation source is adapted to provide a continuous energy output during treatment of the affected skin, with a momentary output peak power smaller than 10 kW/cm².

32. Apparatus for treatment of skin disorders, comprising:
a radiation source, adapted to generate ultraviolet (UV) radiation suitable for treatment of skin affected by one or more of the skin disorders; and
radiation delivery optics, coupled to the radiation source so as to concentrate and deliver the generated UV radiation directly to the affected skin with an energy flux level per single treatment and specific affected skin area selected from an energy radiation flux group consisting of at least a radiation flux equal or higher than 50 mJ/cm² in less than 30 sec per treatment and a radiation flux higher than 1.5 MED (Minimum Erythema Dose),
wherein the UV radiation is in a spectral range of 296-390 nm and comprises at least one spectral line of substantial intensity having a bandwidth of at least 1 nm, wherein the spectral line is selected so as to engender clearing of the affected skin, and
wherein the apparatus is adapted to determine the radiation flux corresponding to the MED by determining a minimum energy dosage creating erythema on normal skin in a vicinity of the affected skin area, whereby operating parameters of the apparatus are set to the radiation flux thus determined.

33. Apparatus for treatment of skin disorders, comprising:
a radiation source, adapted to generate ultraviolet (UV) radiation suitable for treatment of skin affected by one or more of the skin disorders; and
radiation delivery optics, coupled to the radiation source so as to concentrate and deliver the generated UV radiation directly to the affected skin with an energy flux level per single treatment and specific affected skin area selected from an energy radiation flux group consisting of at least a radiation flux equal or higher than 50 mJ/cm² in less than 30 sec per treatment and a radiation flux higher than 1.5 MED (Minimum Erythema Dose),
wherein the UV radiation is in a spectral range of 296-390 nm and comprises at least one spectral line of substantial intensity having a bandwidth of at least 1 nm, wherein the spectral line is selected so as to engender clearing of the affected skin, and
wherein the radiation source further comprises at least a second source of radiation suitable to photothermolyze blood vessels in a vicinity of the affected skin.

34. Apparatus according to claim 33, wherein the second radiation source comprises a pulsed source emitting at least one of visible radiation and near infrared (NIR) radiation.

35. Apparatus for treatment of skin disorders, comprising:
a radiation source, adapted to generate ultraviolet (UV) radiation suitable for treatment of skin affected by one or more of the skin disorders; and
radiation delivery optics, coupled to the radiation source so as to concentrate and deliver the generated UV radiation directly to the affected skin with an energy flux level per single treatment and specific affected skin area selected from an energy radiation flux group consisting of at least a radiation flux equal or higher than 50 mJ/cm² in less than 30 sec per treatment and a radiation flux higher than 1.5 MED (Minimum Erythema Dose),
wherein the UV radiation is in a spectral range of 296-390 nm and comprises at least one spectral line of substantial intensity having a bandwidth of at least 1 nm, wherein the spectral line is selected so as to engender clearing of the affected skin, and
wherein the radiation source comprises a metal halide lamp, whose output spectrum comprises spectral lines at 303, 306, 308, 309, and 312 nm.

36-42. (canceled)

43. Apparatus for treatment of skin disorders, comprising:
a radiation source, which is adapted to generate radiation in multiple spectral bands;
a radiation guide, which is optically coupled to receive the radiation in all of the multiple spectral bands, and to convey the received radiation to an area of skin affected by one of the disorders, so as to treat the affected area; and
a band selector, which is adapted to select one or more of the multiple spectral bands to be conveyed by the radiation guide, in response to a therapeutic indication,
wherein the multiple spectral bands comprise at least one band in each of an ultraviolet A (UVA) range, an ultraviolet B (UVB) range, and a visible range.

44. Apparatus according to claim 43, wherein the visible range comprises a violet light range.

45. Apparatus according to claim 43, wherein the visible range comprises a red light range.

46. Apparatus according to claim 43, wherein the band selector is adapted to select at least two of the spectral bands to be conveyed by the radiation guide simultaneously.

47. Apparatus according to claim 46, wherein the at least two of the bands comprise the at least one band in one of the UVA and UVB ranges and the at least one band in the visible range.

48. Apparatus according to claim 47, wherein the at least one band in the one of the UVA and UVB ranges comprises one or more wavelengths in the UVB range that provide effective treatment of a psoriatic plaque, while the at least one band in the visible range comprises violet light suitable for treating inflammation associated with the plaque.

49. Apparatus for treatment of skin disorders, comprising:
a radiation source, which is adapted to generate radiation in multiple spectral bands;
a radiation guide, which is optically coupled to receive the radiation in all of the multiple spectral bands, and to convey the received radiation to an area of skin affected by one of the disorders, so as to treat the affected area; and
a band selector, which is adapted to select one or more of the multiple spectral bands to be conveyed by the radiation guide, in response to a therapeutic indication,
wherein the radiation source comprises a plurality of lamps, each operating in one or more of the spectral bands, and a beam combiner for combining the radiation from the plurality of the lamps to be received by the radiation guide.

50. Apparatus according to claim 49, wherein the plurality of lamps comprise electrical discharge lamps.

51. Apparatus according to claim 49, wherein the plurality of lamps comprise solid-state light sources.

52. Apparatus according to claim 49, wherein the plurality of lamps comprise at least one electrical discharge lamp and at least one solid-state light source.

53. Apparatus according to claim 49, wherein the beam combiner comprises one or more dichroic mirrors, which are adapted to selectively reflect the radiation emitted by the lamps.

54. Apparatus according to claim 49, wherein the band selector is arranged to actuate one or more of the lamps to operate so as to provide the selected one or more of the spectral bands.

55. Apparatus for treatment of skin disorders, comprising:
a radiation source, which is adapted to generate radiation in multiple spectral bands;
a radiation guide, which is optically coupled to receive the radiation in all of the multiple spectral bands, and to convey the received radiation to an area of skin affected by one of the disorders, so as to treat the affected area; and
a band selector, which is adapted to select one or more of the multiple spectral bands to be conveyed by the radiation guide, in response to a therapeutic indication,
wherein the band selector comprises one or more optical filters, having a selectable spectral passband.

56. Apparatus for treatment of skin disorders, comprising:
a radiation source, which is adapted to generate radiation in multiple spectral bands;
a radiation guide, which is optically coupled to receive the radiation in all of the multiple spectral bands, and to convey the received radiation to an area of skin affected by one of the disorders, so as to treat the affected area; and
a band selector, which is adapted to select one or more of the multiple spectral bands to be conveyed by the radiation guide, in response to a therapeutic indication,
wherein the radiation guide comprises at least one of a fiberoptic light guide and a liquid-filled light guide.

57. Apparatus for treatment of skin disorders, comprising:
a radiation source, which is adapted to generate radiation in multiple spectral bands;
a radiation guide, which is optically coupled to receive the radiation in all of the multiple spectral bands, and to convey the received radiation to an area of skin affected by one of the disorders, so as to treat the affected area; and
a band selector, which is adapted to select one or more of the multiple spectral bands to be conveyed by the radiation guide, in response to a therapeutic indication,
wherein the radiation guide comprises a proximal end, which is coupled to receive the radiation, and a distal end, which is adapted to deliver the radiation to the area of the skin, and further comprising:
a receptacle, for receiving the distal end of the radiation guide;
a detector, which is coupled to receive the radiation emitted from the distal end of the radiation guide when the radiation guide is inserted in the receptacle, and to generate a signal in response to an intensity of the radiation; and
a controller, which is coupled to receive the signal from the detector and to determine, based on the signal, an output level of the apparatus.

58. Apparatus according to claim 57, wherein the controller is adapted to adjust an operating level of the radiation source in response to the signal, so as to adjust the output level to a predetermined value.

59. Apparatus for treatment of skin disorders, comprising:
a radiation source, which is adapted to generate radiation in multiple spectral bands;
a radiation guide, which is optically coupled to receive the radiation in all of the multiple spectral bands, and to convey the received radiation to an area of skin affected by one of the disorders, so as to treat the affected area; and
a band selector, which is adapted to select one or more of the multiple spectral bands to be conveyed by the radiation guide, in response to a therapeutic indication,
and comprising a controller having a user interface, which is operable by an operator of the apparatus to input the therapeutic indication to the band selector.

60. Apparatus according to claim 59, wherein the user interface is further operable by the operator to initiate a procedure, using the apparatus, for determining a Minimal Erythema Dose (MED) of a patient in treatment, and wherein the controller is adapted to set a level of the radiation to be applied to the affected area of the skin of the patient based on the determined MED.

61. Apparatus according to claim 59, wherein the controller comprises a memory, and wherein the user interface is further operable by the operator to record and recall a treatment history of a patient in treatment using the apparatus.

62-67. (canceled)

68. A method for treatment of skin disorders, comprising:
directing radiation in multiple spectral bands toward an input end of a radiation guide;
selecting one or more of the multiple spectral bands to be input to the radiation guide through the input end, in response to a therapeutic indication with respect to an area of skin affected by one of the disorders; and
applying an output end of the radiation guide to the affected area of the skin as to treat the affected area,
wherein the multiple spectral bands comprise at least one band in at least two spectral ranges selected from the group consisting of ultraviolet A (UVA) range, an ultraviolet B (UVB) range, and a visible range, and
wherein selecting the one or more of the multiple spectral bands comprises selecting at least two of the spectral bands to be conveyed by the radiation guide simultaneously, and
wherein the at least two of the bands comprise the at least one band in one of the UVA and UVB ranges and the at least one band in the visible range, and
wherein the at least one band in one of the UVA and UVB ranges comprises one or more wavelengths in the UVB range that provide effective treatment of a psoriatic plaque, while the at least one band in the visible range comprises violet light suitable for treating inflammation associated with the plaque.

69. A method for treatment of skin disorders, comprising:
directing radiation in multiple spectral bands toward an input end of a radiation guide;
selecting one or more of the multiple spectral bands to be input to the radiation guide through the input end, in response to a therapeutic indication with respect to an area of skin affected by one of the disorders; and
applying an output end of the radiation guide to the affected area of the skin as to treat the affected area,
wherein selecting the one or more of the multiple spectral bands comprises receiving an input of the therapeutic indication from an operator, and selecting the one or more of the spectral bands automatically in response to the input.

70. A method for treatment of skin disorders, comprising:
directing radiation in multiple spectral bands toward an input end of a radiation guide;
selecting one or more of the multiple spectral bands to be input to the radiation guide through the input end, in response to a therapeutic indication with respect to an area of skin affected by one of the disorders; and
applying an output end of the radiation guide to the affected area of the skin as to treat the affected area,
wherein directing the radiation comprises determining a Minimal Erythema Dose (MED) of a patient in treatment, and setting a level of the radiation to be applied to the affected area of the skin of the patient based on the determined MED.
